(19) United States
(12) Patent Application Publication
(10) Pub. No.: US 2023/0181921 A1
(43) Pub. Date: Jun. 15, 2023

(54) PHOTOTHERAPY DEVICE

(71) Applicant: The University of Birmingham, Birmingham (GB)

(72) Inventors: David James DAVIES, Birmingham, West Midlands (GB); William Mark PALIN, Birmingham, West Midlands (GB); Mohammed Abdul HADIS, Birmingham, West Midlands (GB)

(21) Appl. No.: 17/922,157

(22) PCT Filed: Apr. 27, 2021
(86) PCT No.: PCT/GB2021/051017

§ 371 Date: Oct. 28, 2022

(30) Foreign Application Priority Data
Apr. 28, 2020 (GB) 2006201.4

(51) Int. Cl. A61N 5/06

(52) U.S. Cl. CPC A61N 5/0601; A61N 2005/0612; A61N 2005/0627; A61N 2005/0647

(57) ABSTRACT

An apparatus for treating TBI or TSI comprises at least two optical fibers and a light source which is operatively connected to at least one of the optical fibres. The apparatus may comprise an adapter for securing the optical fibres to the body of the subject, enabling the direct delivery of light to the brain or spinal cord. Methods and kits for treating TBI or TSI, and methods for determining the effectiveness of treatment are also provided.

The present invention relates to apparatus and methods for treating traumatic brain injury (TBI) or traumatic spinal injury (TSI). More particularly, the present invention relates to apparatus and methods for treating TBI or TSI by delivering light directly to the brain or spinal cord.

Approximately 13 million individuals throughout the European Union and North America are living with the deleterious effects of traumatic brain injury (TBI). Currently the treatment options available to the clinician managing patients who have sustained a TBI are mainly limited to maintaining intra cranial homeostasis, providing optimal conditions for the prevention of secondary brain tissue injury (beyond the initial traumatic insult), and preventing detrimental rises in intra cranial pressure. Currently no disease modifying treatment or intervention exists within TBI care.

In a similar vein, traumatic spinal cord injury (TSI) is also an injury for which no primary intervention for the preservation of cells or neurological function currently exists. Again, the central pillars of management incorporate the stabilisation of the bony spine along with decompression of any active impingement caused by trauma deformity.

The present invention has been devised with these issues in mind.

According to a first aspect of the invention, there is provided an apparatus for treating TBI or TSI, the apparatus comprising:
at least two optical fibres; and
a light source which is optically connected to at least one of the optical fibres, wherein the apparatus is configured to deliver light directly to the brain or spinal cord.

The light may be delivered such that the brain or spinal cord tissue receives an irradiance of from 10 to 90 mW/cm², from 15 to 85 mW/cm², from 18 to 80 mW/cm², from 20 to 60 mW/cm², from 30 to 50 mW/cm², or from 40 to 45 mW/cm², e.g. about 42 mW/cm², or 42.4 mW/cm². Thus, the apparatus may be configured to, in use, deliver an irradiance of from 10 to 90 mW/cm², from 15 to 85 mW/cm², from 18 to 80 mW/cm², from 20 to 60 mW/cm², from 30 to 50 mW/cm², or from 40 to 45 mW/cm² (e.g. about 42 mW/cm², or 42.4 mW/cm²) to the brain or spinal cord. In some embodiments, the light may be delivered such that the brain or spinal cord tissue receives an irradiance of from 18 to 45 mW/cm², or from 20 to 43 mW/cm², e.g. from about 21 to about 42 mW/cm². In some embodiments the apparatus may be configured to, in use, deliver an irradiance of from 18 to 45 mW/cm², or from 20 to 43 mW/cm², e.g. from about 21 to about 42 mW/cm².

It has been found by the present inventors that direct delivery of light to the brain or spinal cord, e.g. at an irradiance of approximately 42 mW/cm² (for example, 42.4 mW/cm²), can result in a significant reduction in hippocampal cell loss to apoptosis, as well as a significant increase in retinal ganglion cell survival following trauma.

The dose of light delivered to the brain or spinal cord tissue is a function of the irradiance and the duration of time the tissue is exposed to the light. In use, the apparatus may be configured to deliver light to the brain or spinal cord tissue for at least 30 seconds, at least one minute, at least 90 seconds, at least two minutes, at least 3 minutes or at least 4 minutes. The apparatus may be configured to deliver light to the brain or spinal cord tissue for no more than 10 minutes, no more than 8 minutes, no more than 5 minutes, no more than 4 minutes, or no more than 3 minutes. In some embodiments, in use the apparatus is configured to deliver light to the brain or spinal cord tissue for a period of time of from 30 seconds to 2 minutes, from 45 seconds to 90 seconds, or from 50 to 80 seconds (e.g. for approximately 1 minute).

Thus, the apparatus may be configured to, in use, deliver a dose of from 1 to 12 J/cm², from 2 to 10 J/cm² or from 3 to 8 J/cm², e.g. from 1.2 to 10.8, or from 2.4 to 7.2, or from 3.6 to 5.4 J/cm².

The light may have a wavelength of from 600 to 1200 nm, from 620 to 1000 nm, from 630 to 800 nm or from 650 to 700 nm, e.g. about 660 nm. The light may be infrared or near infrared light.

Any suitable light source may be used. In some embodiments, the light source is an LED or a laser. In some embodiments, the apparatus comprises multiple light sources (e.g. a plurality of LEDs). For example, one light source may be provided for each of a plurality of optical fibres.

Near infrared light is thought to have significant photobiomodulation capabilities. However, for the light to have any meaningful effect, a specific, concentrated and controllable local irradiance and dose at an intracellular level is thought to be required. The soft and hard tissues of the head and spinal column can scatter, absorb and reflect light, such that only 1-2% of photons delivered externally will reach the target tissue. The present invention mitigates this problem by delivering light directly to the target tissue, either intracranially in the case of TBI treatment, or within the spinal canal in the case of TSI treatment.

According to a second aspect of the invention, there is provided an apparatus for treating TBI or TSI in a subject, the apparatus comprising:
at least two optical fibres;
a light source which is operatively connected to at least one of the optical fibres; and
an adapter for securing the optical fibres to the body of the subject.

The apparatus of the present invention is configured for delivering light directly to the brain or the spinal cord. As used herein, the delivery of light “directly” to the brain or spinal cord refers to the delivery of light intracranially (i.e. within the intracranial space), or intraspinally (i.e. within the spinal canal). This is in contrast to the external delivery of light wherein light has to pass through bone and/or other tissues, or a port therein, in order to reach the tissue to be treated. The present invention therefore enables light therapy (i.e. phototherapy) to be delivered directly to the injured tissue. This overcomes the problem of absorption and diffraction by bone and other tissues when is light delivered externally.

The adapter secures the optical fibres to the body of the subject. This helps to ensure that the desired dose is delivered to the injured tissue, by preventing the optical fibres from moving or becoming detached from the body. In use, the adapter may secure the optical fibres to the body for a period of days, weeks or months. Thus, the fibres may remain in situ in between doses. The optical fibres are secured by the adapter such that light from the light source (which may be external to the body) is delivered directly to the brain or spinal cord, when the apparatus is in use. Thus, the term “adapter”, as used herein, refers to a component which secures the optical fibres to the subject. The adapter may also be referred to as a “fixator”. In some embodiments, the adapter provides an interface though which optical fibres pass before entering the body of the subject.

The apparatus of the first aspect of the invention may further comprise an adapter as described herein. Any features, embodiments or components described in relation to one aspect of the invention may be combined with any other aspect of the invention, unless otherwise stated.

The adapter may be configured to guide the optical fibres into the body of the subject, e.g. into the cranial cavity or the spinal canal. For example, in use, the adapter may be secured to the body of the subject, and then the fibres may be passed along or through the adapter in order to achieve the correct positioning. Alternatively, the fibres may be passed into the body of the subject, and then the adapter fitted to secure the fibres in place.

The adaptor may comprise at least one securing element for releasably attaching the adapter to the body of the subject. The securing element may be any suitable means for releasably attaching the adapter to the body.

In some embodiments, the securing element is a screw thread. Screw threads may be used to attach the adapter to bone, such as the skull.

In some embodiments, the securing element comprises an adhesive portion. For example, the securing element may comprise an adhesive pad, tag or strip. An adhesive portion may be used to attach the adapter to the skin.

In some embodiments, the securing element comprises a loop or aperture (e.g. an islet). Sutures may be passed through the loop, aperture or islet to attach the adapter to the body.

In some embodiments the adapter comprises at least one slot or hole for receiving the optical fibres therethrough. In use, the optical fibres pass through the slot or hole and into the cranium or spinal canal. Each optical fibre may pass through a separate slot or hole. In some embodiments, two or more optical fibres may form a bundle which pass through a slot or hole in the adapter. In some embodiments, some or all of the optical fibres (e.g. in the form of a bundle) pass through the same slot or hole in the adapter.

In some embodiments, the adapter comprises a collar for receiving the optical fibres therethrough.

The adapter may function to maintain the correct positioning of the optical fibres relative to the brain or the spinal cord. Thus, in some embodiments, the adapter comprises a locking element for locking the position of the optical fibres relative to the adapter, when the apparatus is in use. It may be important to lock the position of the optical fibres once they have been inserted into the body of the subject, to ensure that the light therapy is delivered to the injured tissue and/or to prevent damage to neighbouring tissue. The locking element may maintain the correct depth of penetration of the fibres into the cranial cavity or spinal canal, when the apparatus is in use.

In some embodiments, the locking element comprises a screw. For example, the screw may be tightened so as to lock the optical fibres in place as they pass through a hole or slot in the adapter.

In some embodiments the adapter comprises at least two, at least three, at least four or more holes or slots.

In some embodiments the number of holes or slots is equal to the number of optical fibres. In such embodiments, each hole or slot may receive a single optical fibre.

In some embodiments the adapter comprises a greater number of holes or slots than the number of optical fibres. For example, one or more additional holes or slots may be provided. The additional hole(s) or slot(s) may be for receiving a monitoring probe, and/or for securing the adapter to the body of the subject.

The hole(s) or slot(s) may be sized so as to receive the optical fibre(s) and/or a monitoring probe which pass therethrough with a close fit. As such the fibres, and probe if present, are held securely in place by the adaptor.

The adapter may be formed by any suitable technique, such as machining, moulding or 3D printing.

In some embodiments, the apparatus comprises multiple adapters, e.g. two, three or four adapters. Multiple adapters may be useful to treat an injury which extends over a large area of tissue, by securing optical fibres to different points on the subject’s body such that light can be delivered over a wider area.

An integral part of moderate to severe TBI management involves the placement of an invasive intra-cranial monitoring device into the skull of a patient, in order to measure a variety of parameters such as pressure, oxygen tension and extra-cellular biochemistry. This is a universal practice in the majority of specialist units, and is a mandated standard of care in many nations. The present inventors have recognised that the intracranial delivery of light for the treatment of TBI, for example using an adaptor to secure optical fibres which pass into the skull and deliver light directly to the brain, is no more invasive and carries no greater risk than existing monitoring practices, while providing a significantly greater benefit in terms of survival and outcome.

In use, the adapter may be placed into or adjacent an opening (e.g. a burr hole) in the skull, in the case of TBI treatment. Thus, in some embodiments, the adapter is releasably attachable to the skull. In some embodiments, the adapter comprises a screw thread for attaching the adapter to the skull.

The optical fibres, held in place by the adapter, may pass through the opening in the skull. In use, the adapter may be attached to the skull and then the optical fibres may be fed through the hole(s) in the adapter. Alternatively, the optical fibres may extend partially or fully through the hole(s) in the adapter before the adapter is attached to the skull. In use, the fibres may extend into the cranial cavity, and optionally into the brain tissue, such that the light can be delivered close to the site where injury has occurred. For example, the fibres may be positioned such that their tips are proximal to the site of injury, prior to treatment.

It will therefore be appreciated that, in some embodiments, in use the light source is external to the skull, while the optical fibres pass through the skull (via the adapter), thereby delivering light from the light source directly to the brain.

In some embodiments the adapter comprises a head portion and a screw thread extending from the head portion. The screw thread constitutes the securing element for attaching the adapter to the body. In other words, the adapter is in the form of a bolt. The one or more holes for receiving the optical fibres may extend through the head portion and the screw thread of the adapter. In use, the adapter may be screwed into a burr hole in the skull of the subject, and the optical fibres passed through the hole(s) in the adapter. The fibres may then be inserted such that they extend into the brain tissue, or the fibre tips may be positioned adjacent the brain surface, depending on the site of injury.

The adapter may be formed from any suitable material. For example, for insertion into the skull for treatment of TBI, it will be appreciated that the adapter will require structural integrity.

In some embodiments, the adapter is formed, entirely or in part, from a biocompatible material.

In some embodiments, the adapter is formed, entirely or in part, from plastic, such as nylon or polyethylene (e.g. high density polyethylene (HDPE)). In some embodiments, the adapter is formed, entirely or in part, from metal, such as stainless steel or titanium.

In some embodiments, the adapter comprises a coating. The coating may be biocompatible. In some embodiments, the coating comprises an antimicrobial agent.

In the case of spinal cord treatment, the optical fibres may be passed between adjacent vertebrae into the spinal canal, so as to deliver light to one or more regions of spinal tissue directly.

In some embodiments, e.g. for the treatment of TSI, the adapter may comprise a collar for receiving the optical fibres therethrough.

The collar may be formed from any suitable material. For example, the collar may be formed, entirely or in part, from plastic, such as nylon or polyethylene (e.g. high density polyethylene (HDPE)) or metal, such as stainless steel or titanium.

The collar may be provided with a securing element for attaching to the skin. The securing element may be attached to the collar, or integrally formed with the collar. In some embodiments, the collar is mounted on the securing element.

In some embodiments, the securing element comprises one or more tags. The tags may have apertures therein. The apertures may be used to attach the adapter to the skin, for example using sutures. Alternatively, the securing element may comprise an adhesive portion, e.g. adhesive tags, or an adhesive pad or strip, for attaching the adapter to the skin.

A securing element for attaching the adapter to the skin may advantageously be formed from a flexible material.

In some embodiments, the adapter further comprises a locking element for locking the position of the optical fibres within the collar. The locking element may be in the form of a screw which passes through the collar. The screw may be tightened to secure the optical fibres in position, thereby locking the depth to which the optical fibres penetrate into the body.

The collar may comprise a proximal surface, which is positioned closest to the skin in use, and a distal surface. In some embodiments, the distal surface of the collar is provided with one or more connectors. The connectors may enable the transmission of light between the optical fibres and a light source.

The light source may be external to the body, when the apparatus is in use. Any suitable light source may be used. In some embodiments, the light source is an LED or a laser.

In some embodiments, the apparatus comprises multiple light sources (e.g. a plurality of LEDs). For example, one light source may be provided for each of a plurality of optical fibres.

The light source may be configured to emit light having a wavelength of from 600 to 1200 nm, from 620 to 1000 nm, from 630 to 800 nm or from 650 to 700 nm, e.g. about 660 nm.

It will be appreciated that the optical fibre(s) may be directly connected to the light source, or they may be indirectly connected to the light source, for example via a connector (e.g. a SMA fibre optic connector) or a patch cable.

The apparatus according to the invention comprises at least two optical fibres. At least one of the optical fibres is employed, in use, as a delivery fibre for delivering light to the injured tissue, the delivery fibre being connected to the light source. Fibres which are employed to deliver light and which are connected to the light source may be referred to as “delivery fibres”.

In some embodiments, at least 25%, at least 50% or at least 75% of the optical fibres are delivery fibres. In some embodiments, all of the optical fibres are delivery fibres.

The optical fibres may be single core or multicore fibres. It will be understood that the term “multicore”, as used herein, refers to a single fibre that is split into multiple strands.

A multicore optical fibre may comprise one or more multiple corded strands, i.e. wherein one or more strand is sub-divided into multiple strands.

The optical fibres may have a diameter of from 500 to 3000 µm, from 1000 to 2000 µm or from 1500 to 1800 µm.

In some embodiments, the apparatus comprises a plurality (i.e. an array) of optical fibres. For example, three, four, five, six, seven, eight, nine, ten or more fibres may be provided. The apparatus may comprise at least 4, and least 5, at least 6, at least 8 or at least 10 optical fibres. In some embodiments, the apparatus comprises from 2 to 50, from 5 to 40 or from 10 to 30 optical fibres. The use of a plurality of fibres may help to improve the distribution of light to the target tissue. This may be particularly beneficial for the treatment of an injury which has occurred over a large area of tissue.

In some embodiments, some or all of the optical fibres are bundled together. In some embodiments, some or all of the optical fibres are bonded together with a bundle.

In some embodiments, at least one of the optical fibres is employed, in use, as a detection fibre for detecting the amount of light received by the brain or spinal cord in use. Fibres which are employed to detect light may be referred to as “detection fibres”. The detection fibres are not connected to an external light source.

In use in the treatment of TBI, the detection fibres may be passed deeper into the brain tissue than the delivery fibres. This enables the detection fibres to monitor the dose of light that has been delivered to a particular volume of brain tissue.

The delivery fibres may be structurally the same as the detection fibres.

In some embodiments the apparatus comprises an even number of optical fibres, wherein half of the fibres are employed as delivery fibres, and half of the fibres are employed as detection fibres.

The holes in the adapter, and thus the optical fibres which pass through them, may be arranged in any convenient layout. In some embodiments, the holes are arranged such that, when the apparatus is in use, the optical fibres deliver overlapping beams of light.

In some embodiments, the apparatus further comprises a cap or cover for the adaptor. The cap or cover may be configured for mounting on the adapter, in use. The cap or cover may be configured to cover a portion of the adapter, or the whole of the adapter, when in use.

The cap or cover may comprise a screw thread for attaching to the adapter. Alternatively, the cap or cover may be secured to the adapter via a push fit.

In some embodiments, the cap or cover comprises a light source and/or a photodiode. For example, a light source and a photodiode may be housed within the cap or cover. This enables the light source to be located in close proximity to the body while the apparatus is in use, avoiding the need for long cables to transmit the light.

The cap or cover may comprise one or more SMA connectors for connecting the optical fibres to the light source and/or photodiode. Alternatively, the cap or cover may comprise one or more push-fit couplings for connecting the optical fibres to the light source and/or photodiode.

The cap or cover may comprise a power supply. The power supply may be a cable for supplying power from an external source. Alternatively, the cap or cover may comprise an internal power source, such as a battery.

In some embodiments the apparatus further comprises a probe for monitoring at least one of intracranial pressure, oxygen tension and extra-cellular biochemistry.

The apparatus may further comprise a power source (e.g. a battery).

The apparatus may further comprise one or more patch cables and/or connectors. Patch cables and/or connectors may be used to transport light from the light source(s) to the optical fibre(s).

The apparatus may further comprise one or more additional components selected from lenses, diffusers, couplers, connectors and optical devices, which may be used to control or guide the light delivered by the apparatus when in use.

In some embodiments, the apparatus further comprises one or more spectrometers (e.g. a UV-Vis, Raman and/or Infra-Red spectrometer) and/or a power meter (e.g. a high acquisition rate power meter) to which the detection fibre(s) are connected. This enables the detection and/or monitoring of the light received by the region of tissue of the brain or spinal cord being treated.

For example, some or all of the detection fibre(s) may be used to detect the light being received by the region of tissue being treated, e.g. by detecting the light that has reached the tissue from the delivery fibres. In this way, the detection fibre(s) can be used to determine whether the correct dose has been administered. In such embodiments, at least one detection fibre is connected to a spectrometer, such as a UV-Vis spectrometer, or to a power meter (e.g. a high acquisition rate power meter).

Additionally or alternatively, some or all of the detection fibres may be used to determine whether or not the treatment is effective. This can be achieved by obtaining a spectrum of the injured tissue, e.g. a Raman spectrum. The spectrum obtained may depend on the molecular content of the tissue, which in turn may change depending on the extent of injury (or repair). In such embodiments, at least one detection fibre is connected to a Raman spectrometer.

In some embodiments, one or more detection fibres are connected to a UV-Vis spectrometer, and one or more detection fibres are connected to a Raman spectrometer.

The apparatus may further comprise a controller (e.g. a computer). The controller may be configured to control the operation of the apparatus in use. For example, the controller may be configured to control the intensity, timing and/or duration of the light delivered. The controller may comprise a user interface. The user interface may enable an operator to input treatment parameters, such as the dosage and number of doses required. Optionally, the user interface enables the operator to observe signals detected by the detection fibres.

In some embodiments, the controller is configured, in use, to modulate the light delivered to the brain or spinal cord by the delivery fibres. The controller may be configured to modulate the light delivered in response to the light detected by the detection fibre(s), in use. In this way, the apparatus is able to detect the light received by the tissue and, in real-time, adjust the light delivered such that a therapeutic dose is achieved.

In some embodiments, the controller is configured, in use, to modulate the light delivered to the brain or spinal cord by the delivery fibres in response to a spectrum obtained by the (or each) spectrometer, or in response to a reading obtained by the power meter.

In some embodiments, the controller is configured, in use, to detect a change in a spectrum (e.g. a Raman spectrum) obtained from the treated tissue relative to a reference spectrum. In particular, the controller may be configured to detect a change in the peaks observed in the 1400-1500 cm⁻¹ region and/or the 1600-1700 cm⁻¹ region of a Raman spectrum, relative to the peaks in a corresponding region of a reference spectrum. For example, the ontroller may be configured to detect a change in the spectrum at approximately 1440 cm⁻¹ and/or approximately 1660 cm⁻¹. In some embodiments, the controller is configured, in use, to determine the 1440/1660 cm⁻¹ ratio in a Raman spectrum obtained from the treated tissue, as described herein below. Any changes detected by the controller, and/or the 1440/1660 cm⁻¹ ratio determined by the controller, may be output to the user, e.g. via a user interface. The output provided by the controller may enable the user to determine whether or not further therapy (e.g. the administration of one or more further doses of light) is required.

In some embodiments, for example when an operative intervention following TBI is required which involves opening of the skull, an adapter is not required for locating the optical fibres in the brain. Instead, the optical fibres may be manually placed either at the cortical surface or into the brain parenchyma. This may be achieved by tunnelling a fibre bundle through a small hole in the scalp. This makes extraction of the optical fibres possible without re-opening the craniotomy wound, akin to the way in which any wound drain is removed following surgery. In some embodiments, the fibres extend from a single fibre bundle. This facilitates removal of the fibres from the brain or spine, by simply pulling out the fibre bundle in the same way as removing a surgical drain.

Similarly, if following a TSI a surgical decompression is required (i.e. to remove any active impingement of the spinal cord caused by deformity of the spine), optical fibres may be inserted into the spinal canal without the requirement for an adaptor fixed to the vertebrae.

In some embodiments, a plurality of magnetic datum points may be provided along the length of one or more (or each) of the optical fibres. This would enable the position of the fibre(s) to be determined without the potential difficulties associated with X-ray or CT imaging.

In some embodiments, a portion of each of the fibres is embedded in a carrier material, such as a mesh, pad or membrane. The use of a carrier material may help to support the optical fibres, and hold them in position in close proximity to the tissue to be treated.

The carrier material may be made from a biocompatible material. The carrier material is preferably flexible. The carrier material may be made from a dural substitute material. Suitable materials include collagen, silicone, and gelatine film. In use, the carrier material may be placed onto the surface or the brain, or within the spinal canal.

The optical fibres may be arranged in a radial pattern in the carrier material. This facilitates removal of the fibres.

According to a further aspect of the invention, there is provided a kit for treating TBI or TSI, the kit comprising:
at least two optical fibres;
a light source; and
instructions for use.

The kit may further comprise an adapter as described herein. In some embodiments, the kit comprises multiple adapters, e.g. 2, 3, 4 or more adapters.

The kit may further comprise one or more additional components selected from: a power source, one or more spectrometers (e.g. a UV-Vis, Raman and/or Near Infrared spectrometer), a power meter, one or more patch cables or connectors, a controller (e.g. a computer), a monitoring probe, one or more photodiodes, one or more lenses, one or more diffusers, or any combination thereof.

In some embodiments, the kit further comprises an introducer tool (e.g. a needle), for inserting the optical fibres into the body of the subject.

In some embodiments, the kit further comprises a fibre cutter, for cutting the optical fibres to the required length.

According to a further aspect of the invention, there is provided a method for the treatment of TBI or TSI, the method comprising delivering light directly to the brain or spinal cord of a subject.

As used herein, the term “treatment” will be understood as the partial or complete alleviation of symptoms, a prevention or reduction in cell or tissue death, a reduction in the number or percentage of apoptotic cells, or the rate of apoptosis, improving or retaining cognitive abilities, improving or retaining motor and/or sensory functions, preventing or reducing cognitive impairment, disability and/or paralysis, reducing or eliminating pain, or an improvement in clinically measurable parameters such as reducing or eliminating the need for surgery, reducing the duration of time in hospital, an improvement in brain metabolic measurements, or a reduction in the intensity of therapy required to maintain intracranial homeostasis, or any combination thereof.

The subject may be an animal, such as a horse, cow, pig, goat, dog, cat, primate, or rodent. In some embodiments the subject is human.

In some embodiments, the method comprises delivering light in an amount (i.e. at a dose) which is sufficient to reduce or prevent cell death (apoptosis) or promote cell survival.

The method may comprise delivering an irradiance of light of from 10 to 90 mW/cm², from 15 to 85 mW/cm², from 18 to 80 mW/cm², from 20 to 60 mW/cm², from 30 to 50 mW/cm², or from 40 to 45 mW/cm², e.g. about 42 mW/cm², or 42.4 mW/cm²to injured tissue. In some embodiments, the method comprises delivering an irradiance of light of from 18 to 45 mW/cm², or from 20 to 43 mW/cm², e.g. from about 21 to about 42 mW/cm².

The light may be delivered for at least 30 seconds, at least one minute, at least 90 seconds, at least two minutes, at least 3 minutes or at least 4 minutes. In some embodiments, the light is delivered for no more than 10 minutes, no more than 5 minutes, no more than 4 minutes, or no more than 3 minutes. In some embodiments, the light is delivered for a period of from 30 seconds to 2 minutes, from 45 seconds to 90 seconds, or from 50 to 80 seconds (e.g. for approximately 1 minute). Each period of delivery provides a single “dose”. Within a single dose, the light may be delivered continuously, or it may be pulsed.

The dose delivered to the subject may be from 1 to 12 J/cm², from 2 to 10 J/cm² or from 3 to 8 J/cm², e.g. from 1.2 to 10.8, or from 2.4 to 7.2, or from 3.6 to 5.4 J/cm².

One or more doses (e.g. 2, 3, 4, 5, 6, 7, 8, 9, 10 or more doses) may be delivered to the subject. The dose(s) may be delivered on a number of consecutive days. For example, the subject may receive one or more doses per day for a period of at least 1, 2, 3, 4, 5, 6 or 7 consecutive days, e.g. at least 3 consecutive days. In some embodiments, the method comprises delivering a daily dose of light to the subject for at least 3 or at least 4 consecutive days. In some embodiments, one or more doses are administered to the patient per day for a period of at least 1, 2, 3 or 4 weeks.

The method may comprise delivering a dose of light to the subject once, twice, three, four or more times per day. In some embodiments, one dose is delivered per day. For example, one dose per day may be delivered on at least 2 consecutive days. For example, one dose per day may be delivered on 2, 3, 4, 5 or more consecutive days.

In some embodiments, only a single dose is delivered to the subject.

Treatment may be initiated within 24 hours, 16 hours, 12 hours, 8 hours, 4 hours or 1 hour of injury, or within 45 minutes, 30 minutes or 15 minutes of injury. Preferably, the (or the first) dose of light is delivered to the subject substantially immediately after injury. Administering treatment as soon as possible following injury may help to prevent further cell death.

The light may delivered using an apparatus or kit as described herein.

In some embodiments, the method comprises:
providing at least two optical fibres each having a distal end and a proximal end, wherein the proximal end of at least one of the optical fibres is operatively connected to a light source;
passing the distal ends of the optical fibres into the cranial cavity or spinal canal until the distal ends of the optical fibres are within or proximal to a region of tissue to be treated;
securing the optical fibres to the body of the subject, optionally using an adapter; and
delivering light through at least one of the optical fibres to the tissue to be treated.

The adapter may be as described herein.

The distal ends of the optical fibres may be directed to the target tissue (i.e. the tissue to be treated) using standard imaging techniques, such as computerized tomography (CT) scanning, magnetic resonance imaging (MRI), ultrasound, angiography, radionucleotide imaging, electroencephalography (EEG) and the like.

In some embodiments, the method may comprise a guiding the optical fibres to the target tissue using a stereotactic system.

The principle of stereotactic guidance is widespread within the field of neurosurgery, although it is not generally used for the targeting of intervention for trauma-related pathology. The technique involves the incorporation of a patient-specific set of axial images (e.g. CT or MRI) to fixed datum points on the patient, with or without the use of a frame. This allows the use of imaging to guide the safe placement of instrumentation.

In some embodiments, the method comprise monitoring the light received by the tissue (e.g. the target or injured region) of the brain or spinal cord. This may include determining whether a sufficient or optimal dose has been delivered to the tissue. The light received may be monitored using one or more detection fibres. The detection fibre(s) may be connected to a spectrometer (e.g. a UV-Vis, Raman or Near Infra-red spectrometer), a power meter or a computer. In some embodiments, the detection fibre(s) are connected to a UV-Vis spectrometer.

In some embodiments, the method further comprises modulating the light delivered such that a desired dose (e.g. a therapeutic dose) is received by the target tissue. The amount of light delivered may be modulated in real time.

For example, if it is determined that an insufficient or sub-optimal dose has been delivered, treatment (i.e. the delivery of light) may be continued for an additional period of time until the desired dose has been received by the tissue. Additionally or alternatively, a separate, further dose may be administered. The further dose may be the same as the dose previously administered (i.e. light at the same irradiance level, for the same duration of time), or it may be different to the dose previously administered. For example, the dose may be increased by administering light at a different irradiance level and/or a for a longer duration of time.

As described above, one or more of the optical fibres may serve as a detection fibre, i.e. as a detector of light. The intensity of the light detected by the detection fibre(s), together with the known distance between the delivery fibre(s) and the detection fibre(s), may be used to modulate the light delivered until the desired dose is received by the target tissue. The detection fibres may thus provide a feedback loop which aids in the delivery of the correct dose.

The detection may take the form of optical power detection or spectroscopic analysis. In the case of the optical power detection, extrapolation of the distances between the delivery and detection fibres, together with known optical and anatomical parameters, may be used to achieve a real time modulation of power to provide as optimal a dose to the desired target tissue as is desired. An example of such a method is provided herein. In a broader target area where multiple delivery fibres are employed, computational reconstruction of the power parameters may be used.

In some embodiments, the method further comprises determining the effectiveness of the treatment. For example, the subject may be assessed after each dose of light has been administered (e.g. immediately after each dose), and/or after multiple (e.g. 2, 3, 4, 5) doses, to determine whether the treatment has been effective.

It may be that the method comprises determining whether, and/or the extent to which, the treatment delivered has prevented or reduced apoptosis.

In some embodiments, the method comprises obtaining a spectrum of the tissue before and/or after treatment. In some embodiments, the method comprises obtaining a spectrum (e.g. a Raman spectrum) before treatment, for example to provide a reference spectrum. In some embodiments, the method comprises obtaining a spectrum (e.g. a Raman spectrum) after treatment.

It may be that a spectrum is obtained immediately after the administration of the (or each) dose of light to the subject. Additionally or alternatively, the method comprises obtaining one or more spectra at predetermined time points after dosing (i.e. while therapeutic light is not being delivered). For example, it may be that spectra are obtained at regular intervals (e.g. every 1-4 hours for 24 hours) following dosing, or at specific time points, e.g. 1, 4, 8 and 12 hours, following dosing. In some embodiments, a spectrum is obtained before treatment, immediately after a dose of light has been administered and, optionally, at one or more predetermined time points after the dose. The spectrum may be obtained using the detection fibre(s).

In some embodiments, determining the effectiveness of the treatment comprises detecting a change in a spectrum obtained from the treated tissue, relative to a reference spectrum.

In some embodiments, the spectrum is a Raman or an infrared spectrum. Preferably, the spectrum is a Raman spectrum. Raman spectroscopy produces chemically specific optical signatures by utilising the inelastic scattering of coherent light, detecting the shift in wavelengths (cm⁻¹) observed following the energy change between light and matter. Incident photons vibrate the molecules forming the target substance before scattering and the recaptured photons can be collected to form a spectrum of peaks indicating the biochemical structure of the substance. Raman spectroscopy is sensitive, rapid, achievable without labelling or strict sample preparation, and allows for non-destructive diagnoses or monitoring. It is therefore particularly suitable for intra-cranial monitoring of brain tissue. The present inventors have surprisingly found that changes in the Raman spectroscopic signature elicited by low level light therapy can be utilised to monitor the effectiveness of treatment, and/or as a marker to determine optimal or adequate tissue dosage.

A spectroscope combined with an appropriate interface (e.g. a computer) can be used to observe specific changes in the spectra of the tissue that indicate a successfully ‘treated’ portion of the tissue. In the case of Raman spectra, these changes may be observed in the 1400-1500 cm⁻¹ and/or 1600-1700 cm⁻¹ region. Thus, a shift in the Raman spectrum of the tissue in the 1400-1500 cm⁻¹ and/or 1600-1700 cm⁻¹ region following treatment, as compared to a reference spectrum, may be indicative of effective treatment.

Thus, in some embodiments, the method comprises obtaining a Raman spectrum of the treated tissue. In some embodiments, determining the effectiveness of treatment comprises identifying a change in the spectrum in the 1400-1500 cm⁻¹ region and/or the 1600-1700 cm⁻¹ region as compared to a reference spectrum. In some embodiments, the method comprises identifying a change between the spectrum of the treated tissue and the reference spectrum at approximately 1440 cm⁻¹ and/or approximately 1660 cm⁻¹.

The reference spectrum may be a spectrum of the injured tissue of the subject, obtained prior to treatment. Thus, the method may further comprise obtaining a spectrum of the tissue before treatment is initiated, or before each dose. Alternatively, the reference spectrum may be a spectrum of a portion of healthy tissue, e.g. taken from a healthy portion of the brain or spine of the subject, or taken from a healthy subject.

The present inventors have found that the treatment of injured tissue with low level light therapy, as described herein, results in a change in the ratio of peaks observed at 1440 ± 0.6 cm⁻¹ and 1658 ± 0.6 cm⁻¹ in a Raman spectrum, as compared to untreated control groups. For brevity, this ratio is referred to herein as the “1440/1660 cm⁻¹ ratio”. In treated samples, the 1440/1660 cm⁻¹ ratio was observed to be increased, and closer to 1, relative to the untreated samples. Thus, the value of the 1440/1660 cm⁻¹ ratio, and/or an observed shift in the 1440/1660 cm⁻¹ ratio, may be indicative of effective treatment, or an effective dose.

In some embodiments, the method comprises determining the 1440/1660 cm⁻¹ ratio in a Raman spectrum of the treated tissue. In some embodiments, an increase in the 1440/1660 cm⁻¹ ratio, relative to a reference spectrum obtained prior to treatment, or prior to administration of a dose of light, is indicative of an effective treatment/dose having being delivered to the subject. In some embodiments, a 1440/1660 cm⁻¹ ratio of at least 0.8, 0.85, 0.9 or 0.95 is indicative of effective treatment/dose.

Thus, the present invention provides the use of Raman spectroscopy to determine the effectiveness of direct delivery of light to treat brain and/or spinal cord injuries.

Any of the uses and methods described herein may comprise the administration of further therapeutic interventions to the subject, such as interventions that alleviate intracranial pressure.

Conveniently, the apparatus of the present invention can be used to determine the effectiveness of the treatment, as well as delivering the treatment. This dual utility of the apparatus is particularly advantageous because it means that the use of additional invasive monitoring probes or devices is avoided.

Since the treatment, i.e. the delivery of a dose of light, only occurs for a short period of time (e.g. a few minutes each day), during the remainder of the day the apparatus can be used to monitor the effect of the treatment on the injured tissue, via the detection fibres.

The steps of delivering the treatment, and monitoring the effectiveness of the treatment by obtaining the spectrum of the tissue, may be carried out at different times. This helps to avoid cross-talk between the delivery fibres and the detection fibres. However, cross-talk may be avoided by using a laser as the light source, in which case the delivery of light and monitoring via spectroscopy may be carried out simultaneously.

Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, with any aspect of the invention, unless such features are incompatible. For the avoidance of doubt, the terms “may”, “and/or”, “e.g.”, “for example” and any similar term as used herein should be interpreted as non-limiting such that any feature so-described need not be present. Indeed, any combination of optional features is expressly envisaged without departing from the scope of the invention, whether or not these are expressly claimed. The applicant reserves the right to change any originally filed claim or file any new claim accordingly, including the right to amend any originally filed claim to depend from and/or incorporate any feature of any other claim although not originally claimed in that manner.

Embodiments of the invention will now be described with reference to the accompanying figures, in which:

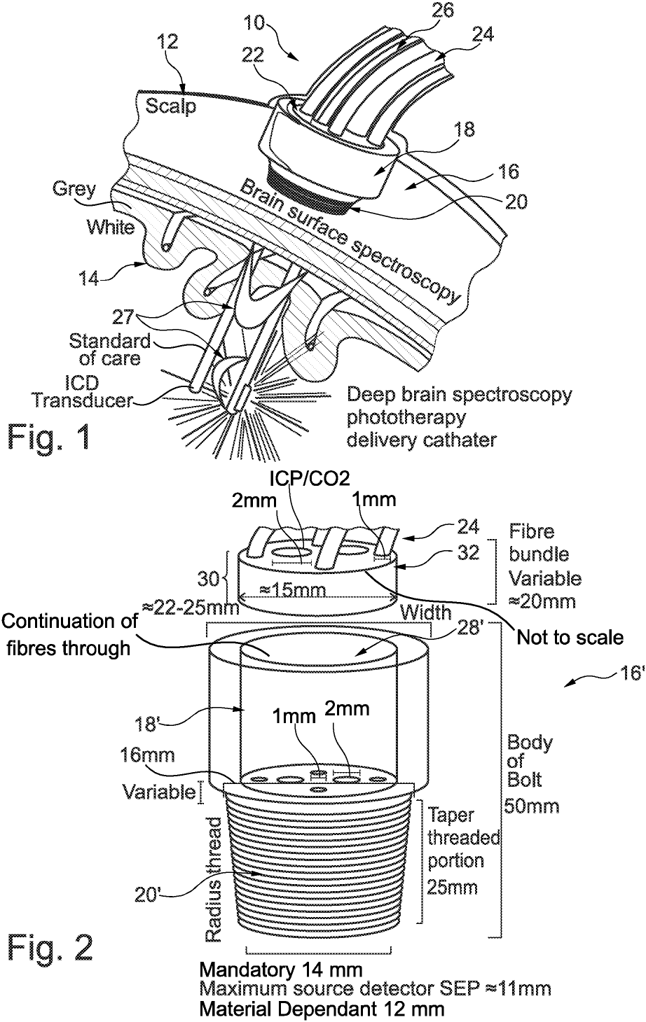

FIG. 1 is an illustration of an apparatus for treating TBI, shown in use;

FIG. 2 is a schematic drawing of a component of an apparatus for treating TBI;

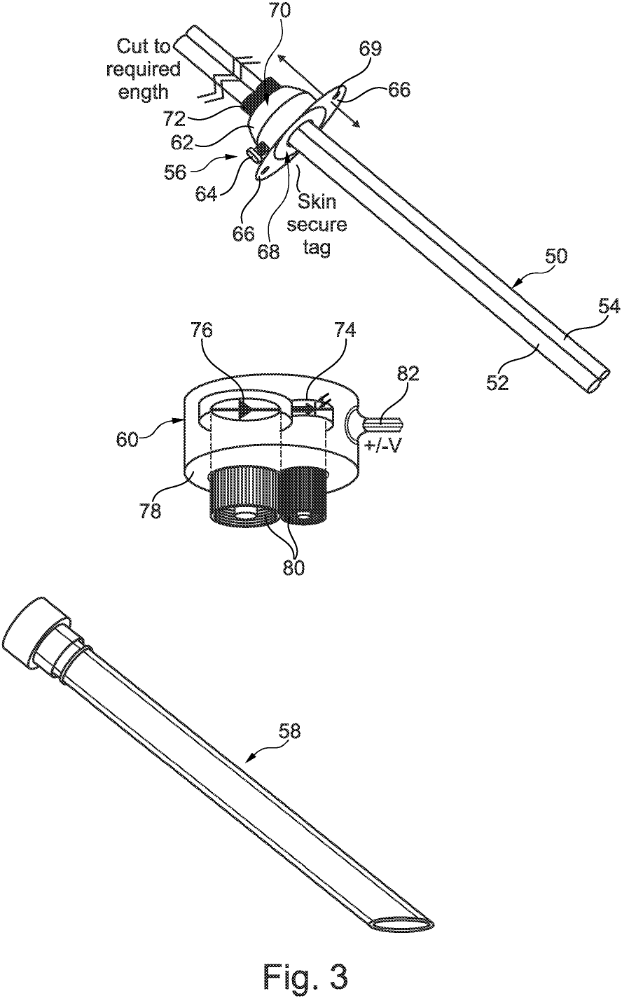

FIG. 3 is an illustration of a kit for treating TSI;

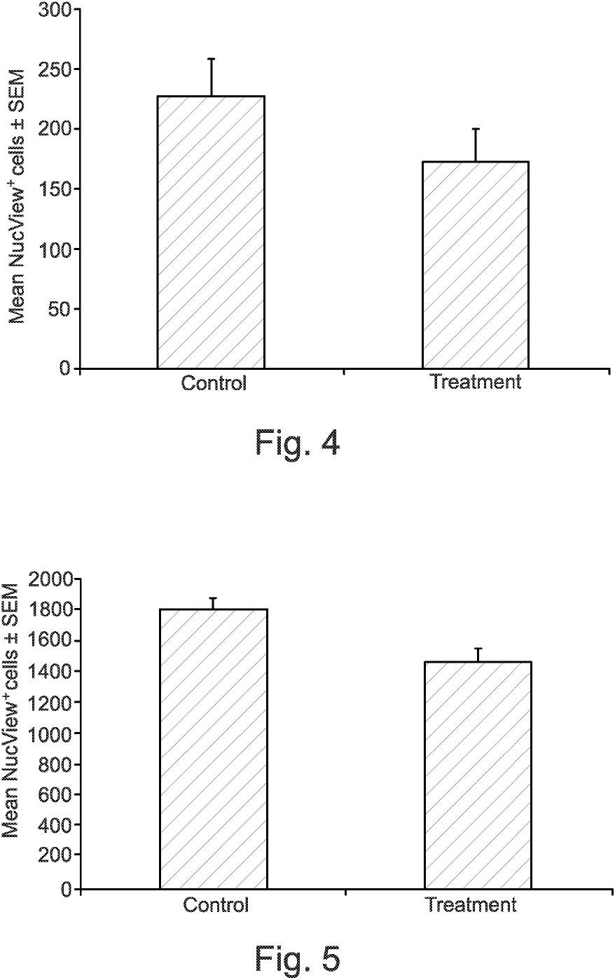

FIG. 4 is a graph showing the number of hippocampal cells entering apoptotic cell death following daily exposure to 660 nm light (Treatment) or ambient light (Control) over 3 days;

FIG. 5 is a graph showing the number of hippocampal cells entering apoptotic cell death following daily exposure to 660 nm light (Treatment) or ambient light (Control) over 5 days;

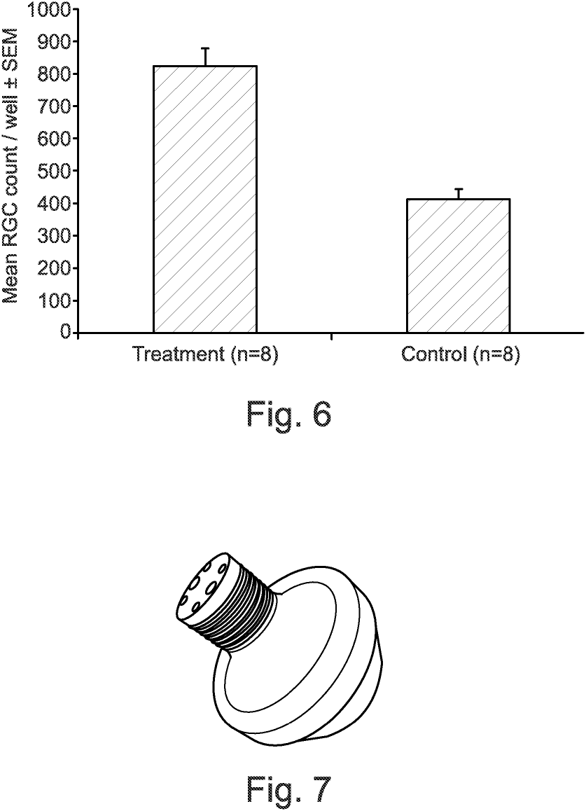

FIG. 6 is a graph showing the number of surviving retinal ganglion cells following daily exposure to 660 nm light (Treatment) or ambient light (Control) over 5 days;

FIG. 7 is a CAD image of a 3D printed adapter for TBI treatment;

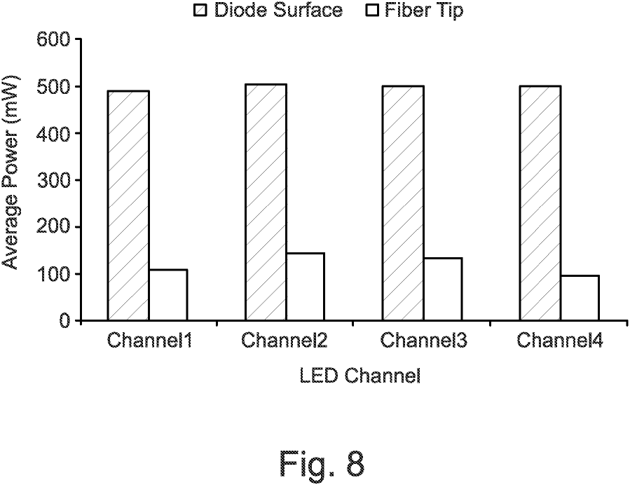

FIG. 8 is a graph showing the average power (mW) recorded over 30 s exposures onto a photodiode power meter at the surface of four LEDs and at the tip exit of coupled optical fibres;

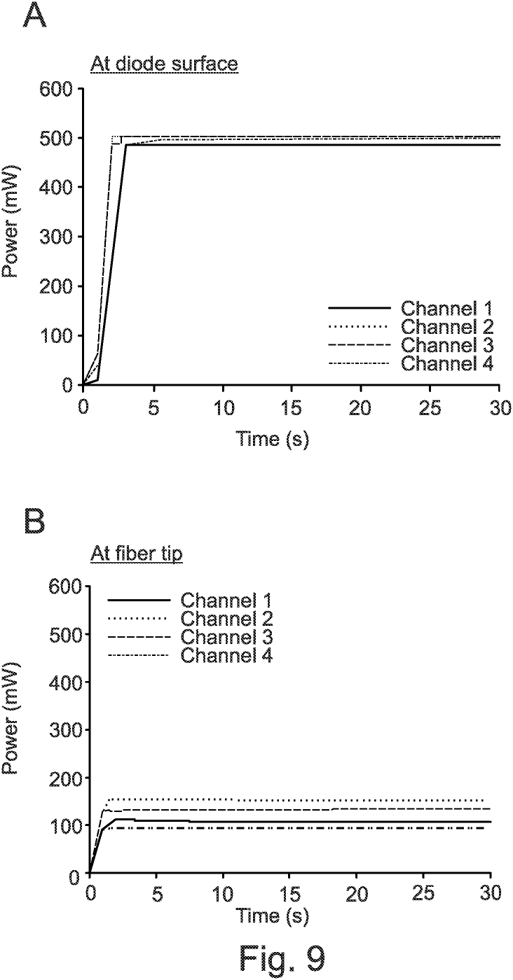

FIG. 9 shows graphs showing the real time power (mW) measured over 30 s exposures onto a photodiode power meter at the surface of four LEDs (FIG. 9A) and at the tip exit of coupled optical fibres (FIG. 9B);

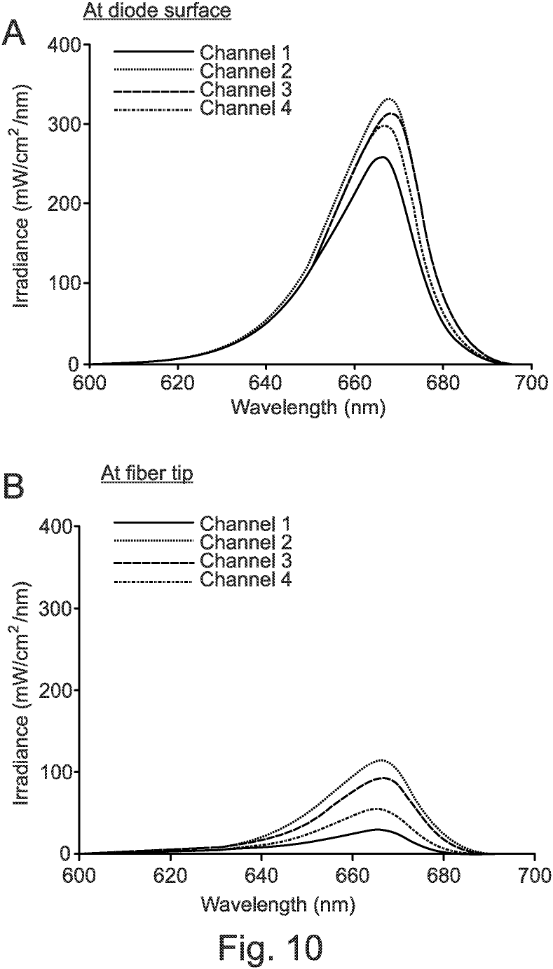

FIG. 10 shows graphs showing the spectral irradiance measured at the LED surface (FIG. 10A) and at a coupled fibre tip (FIG. 10B);

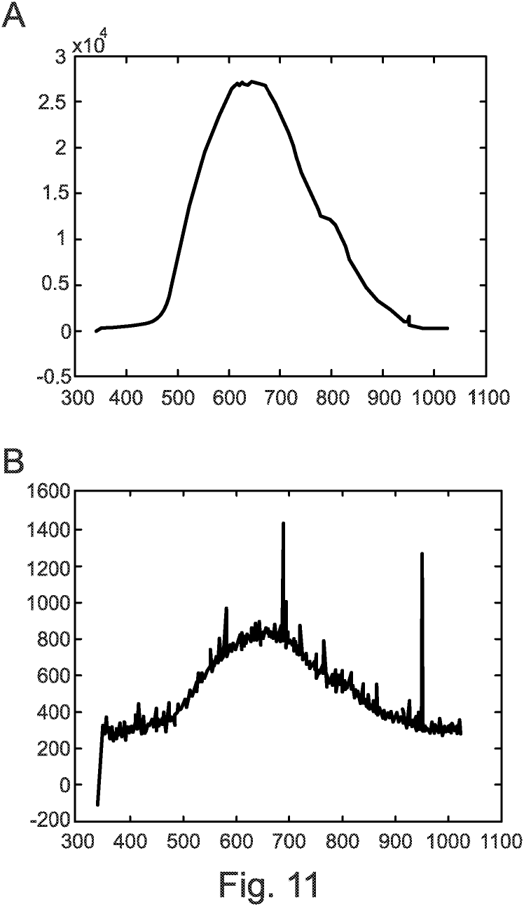

FIG. 11 are broadband spectra (light intensity vs wavelength) detected in a phantom liquid media at 2.4 cm (FIG. 11A) and 1 cm (FIG. 11B) from the internal surface of an adapter. The fibre tips were flush with this surface;

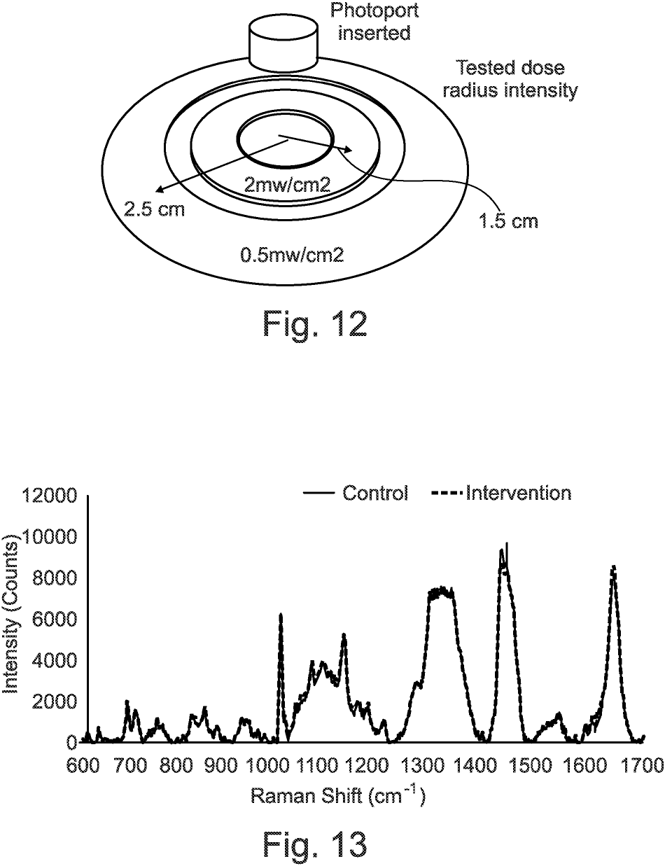

FIG. 12 is a schematic representation of the measured irradiation doses in a cadaveric brain at increasing distances from the internal surface of the adapter;

FIG. 13 shows the effect of 660 nm light on the Raman spectroscopic signature in rat brain tissue;

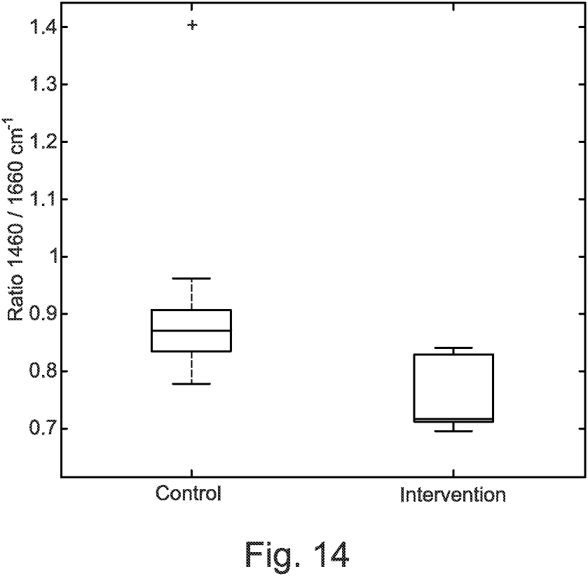

FIG. 14 is a plot showing the change in ratio bands at 1440/1660 cm⁻¹ in the Raman spectra for tissue treated with 660 nm light versus a control;

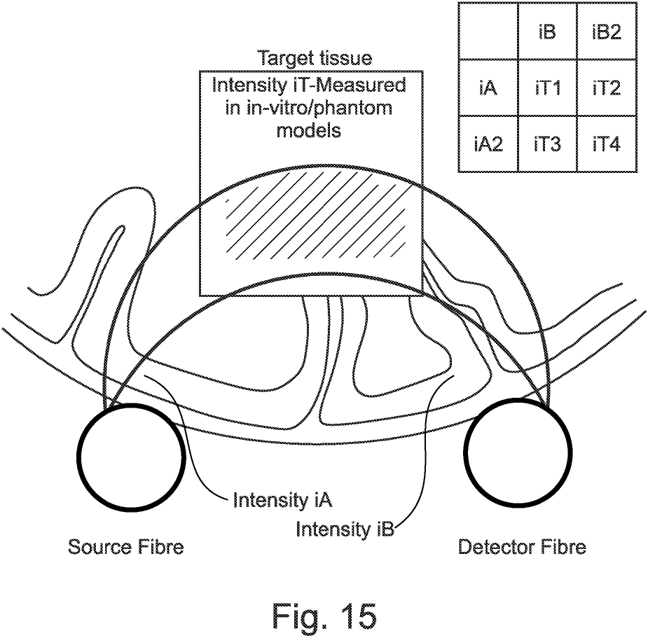

FIG. 15 is a schematic diagram showing how a brain tissue phantom model may be used to formulate data tables which can be used to modulate the dose delivered based on feedback from the light detected;

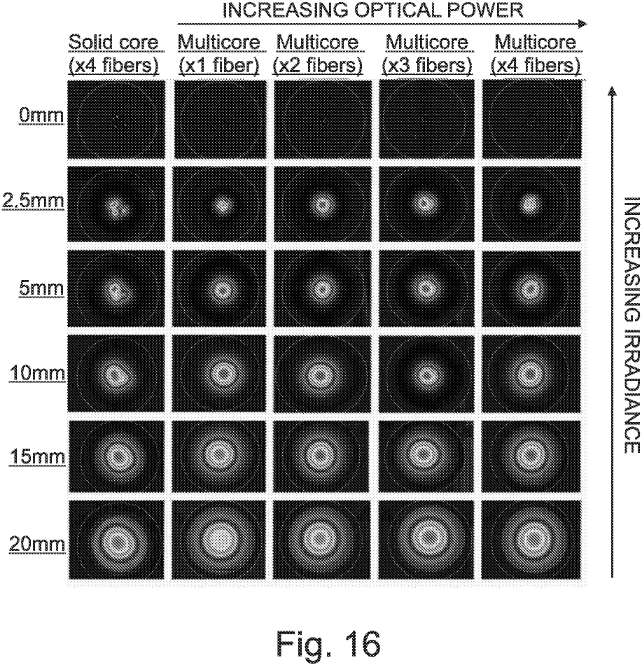

FIG. 16 shows the beam profiles of 660 nm LEDs through phantom media of 2.5 mm - 20 mm thickness. The beam profiles compare the effect of using 4 x solid core fibers (1500 µM each) with up to 4 x multiple core fibres (7 strands x 1000 µM each) on beam distribution;

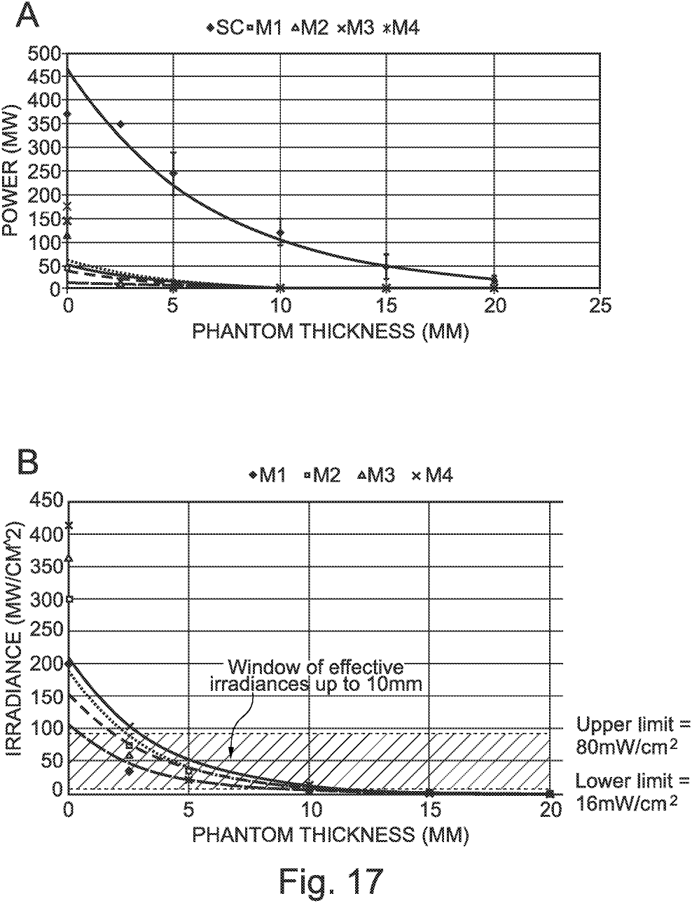

FIG. 17 shows graphs showing the change in (A) power (mW) and (B) irradiance (mW/cm²) of 660 nm LEDs through phantom media of 2.5 mm - 20 mm thickness, where M1-M4 represent increasing number of multi-cored fibres (1-4) and SC represents a solid core fibre. It is evident that for multiple cored fibres, a window of effective irradiances (16-80 mW/cm²) can be achieved between 0-10 mm thickness of phantom media (represented by the shaded area);

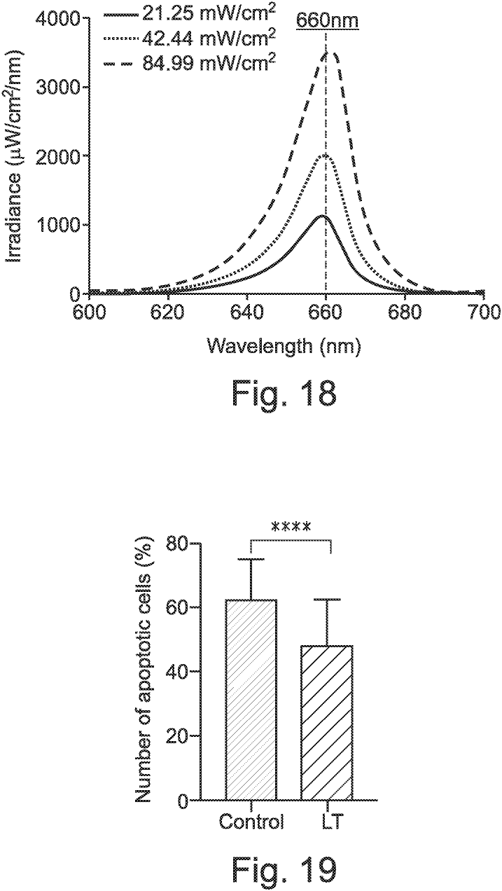

FIG. 18 shows characterisation of the LED array to confirm spectral irradiance and beam homogeneity. Each well is irradiated by 47minature LEDs with Gaussian beam profiles. At the surface of the 6-well plate a homogenous beam profile is produced due to divergence of the beams from the individual LEDs and scattering at the plate surface. This produces a uniform irradiance with an average irradiance of 21.25 mW/cm², 42.44 mW/cm²and 84.99 mW/cm² for the three tested light intensities;

FIG. 19 is a graph showing the initial observed effect in the reduction of cell loss to apoptosis;

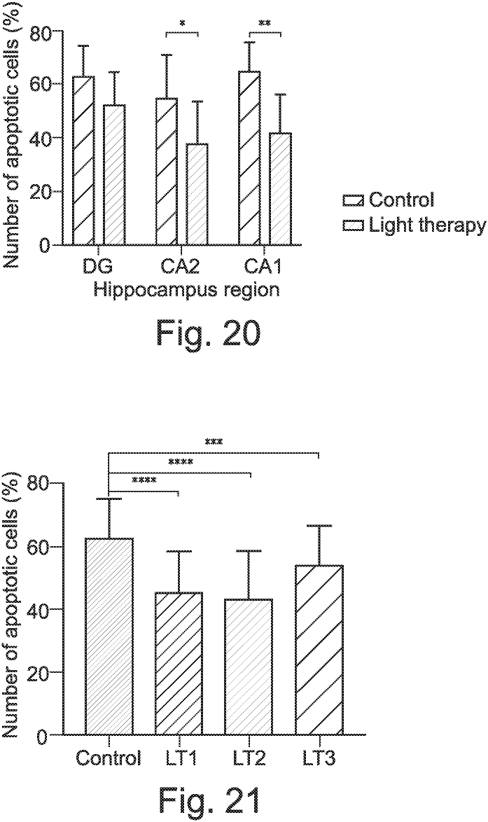

FIG. 20 is a graph showing the differential effect of 660 nm radiation on the three primary regions (DG: dentate gyrus; CA1, CA2: cornu ammonis regions) of observation in the hippocampal sections. ** denotes p<0.001;

FIG. 21 is a graph showing the effect of different levels of irradiance on the percentage of apoptotic cells. LT1 = 21.25 mW/cm²; LT2 = 42.44 mW/cm²; LT3 = 84.99 mW/cm². Error bars show the standard deviation between replicates. **** denotes P<0.0001;

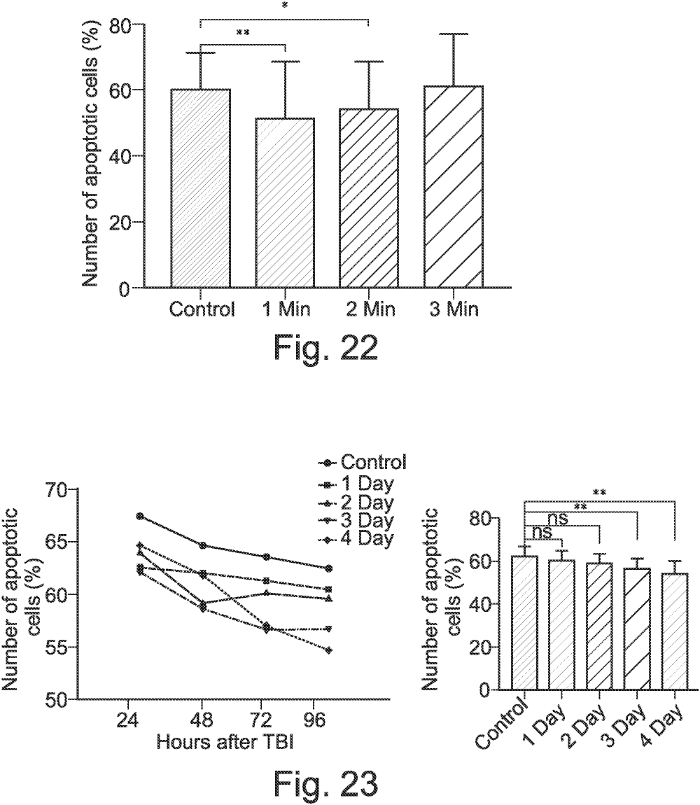

FIG. 22 is a graph showing the effect of dose duration on the percentage of apoptotic cells. ** demotes P<0.001;

FIGS. 23A and 23B are graphs showing the effect of consecutive daily exposure of 42.22 mW/cm² on cell loss to apoptosis. ** denotes P<0.001; and

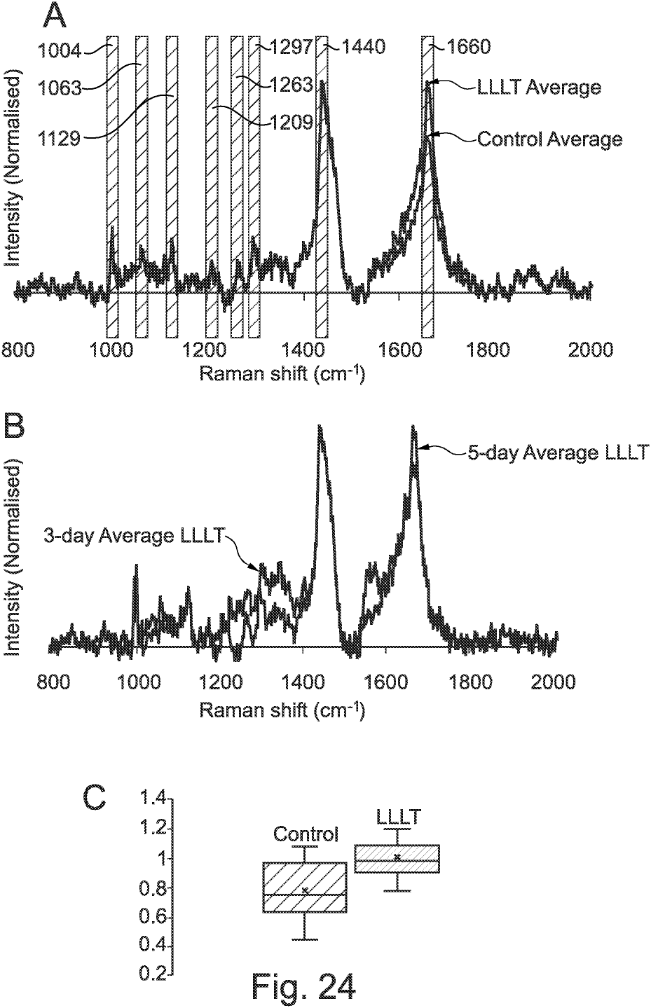

FIG. 24A is a comparative figure of average control and average LLLT Raman spectra normalised, illustrating the change in average intensity of the 1440 cm⁻¹ and 1660 cm⁻¹ peaks, and highlighting the assigned, characteristic peaks of rat hippocampus. FIG. 24B is a comparative figure of normalised Raman spectra from average 3-day LLLT samples and average 5-day LLLT samples. No significant difference was observed between the 1660 cm⁻¹ /1440 cm⁻¹ peak ratios at each therapeutic interval (p= 0.4). FIG. 24C is a Box and Whisker plot illustrating the increase in average peak intensity ratio of 1660 cm⁻¹ / 1440 cm⁻¹, increasing from 0.774 for control samples to 1.002 for LLLT samples (p= 0.0204).

FIG. 1 shows an apparatus in accordance with an embodiment of the present invention. The apparatus comprises an intracranial device (10) located in the skull (12) of a patient for delivering light therapy (e.g. at a wavelength of about 660 nm) directly to the brain (14). The device can be temporarily implanted into the skull in an analogous way to implants used in the monitoring of parameters such as intra-cranial pressure, brain tissue oxygen tension, extra-cellular biochemistry (microdialysis) and occasionally electrophysiological data in TBI patients.

The device comprises an adapter which is releasably attachable to the skull (12). The adapter is in the form of a housing or ‘bolt’ (16) having a head (18) from which extends a screw thread (20) which is screwed into a burr hole in the skull (12). A plurality of holes (22) extend all the way through the bolt (16). Through these holes are passed optical fibres (24) and a standard of care catheter (26), which extend into the brain tissue. In the embodiment shown, four optical fibres are provided, two of which are for delivering light from a light source, such as an LED (not shown), and two of which are for monitoring light received by the brain tissue. However, it will be appreciated that in alternative embodiments different numbers of optical fibres may be provided. The loops (27) emanating from the fibres represent the cross-talk/spectroscopy taking place between the different fibres.

FIG. 2 shows another embodiment of a adapter or ‘bolt’ (16′). In this embodiment, the head (18′) of the bolt is hollowed out to provide a cavity (28). The cavity (28) is sized to accommodate a fibre bundle (30), which comprises a plurality of optical fibres (24) bundled together by a disc-shaped holder (32). When the fibre bundle (30) is received within the cavity (28) of the head (18′) of the bolt (16′), the fibres (24) extend through holes in the screw thread (20′).

In the embodiment shown in FIG. 2, the head (18′) of the bolt (16′) is circular in cross section, having a diameter of about 22-25 mm. The screw thread (20′) is about 25 mm in length, and the overall length of the bolt (i.e. the distance from an upper surface of the head (18′) to an end of the screw thread (20′) is about 50 mm. However it will be appreciated that other dimensions may be suitable.

In addition to the treatment of TBI, low level light therapy may be used to treat traumatic injury to the spinal cord. FIG. 3 shows components of a kit of the present invention which may be used to treat TSI. The kit comprises a bundle (50) of two optical fibres comprising a delivery fibre (52) and a detection fibre (54) which are bonded together, an adapter (56), an introducer tool (58) and a cover (60) for the adapter (56).

The adapter (56) comprises a collar (62) through which the optical fibre bundle (50) passes in use. A grub screw (64) passes through the collar (62), and can be tightened to secure the position of the optical fibre bundle (50) relative to the skin surface, thereby locking the depth to which the optical fibres (52, 54) penetrate into the body of the subject being treated. The adapter (56) further comprises a pair of skin securing tags (66) which extend from a proximal surface (68) (i.e. a skin-facing surfacing) of the collar (62), each having an islet (69) therein so that the tags (66) can be used to secure the adapter (56) to the skin via sutures. Extending from a distal surface (70) of the collar (62) are two connectors (72) for providing an optical connection to the cover (60).

The cover (60) is disc-shaped and houses a photodiode (74) and a light source (76), such as an LED. Extending from a lower surface (78) of the cover (60) are two SMA connectors (80), for mating with the connectors (72) of the adapter (56). Also extending from the cover (60) is a cable (82) for supplying power to the cover (60).

In use, the optical fibre bundle (50) may be inserted into the subject using the introducer tool (58), which is a needle with the central cannula removed so as to provide a hollow conduit through which the optical fibre bundle (50) can be passed. Once the optical fibres (52, 54) have been inserted to the required depth, the introducer tool (58) can be removed, leaving the fibres (52, 54) in place. The collar (62) is threaded over the fibres (52, 54) and secured to the skin using sutures which pass through the islets (68) of the tags (66). The screw (64) is then tightened to lock the fibres (52, 54) at the required depth. The optical fibres (52, 54) are cut to a desired length using an appropriate tool (not shown), which may be provided as part of the kit.

The cover (60) is then fitted such that the connectors (72) on the adapter (56) are received within the SMA connectors (80) of the cover, thereby connecting the delivery fibre (52) to the light source (76) and the detection fibre (54) to the photodiode (74). In the embodiment shown, the SMA connectors comprise internal screw threads which are screwed onto corresponding external screw threads of the connectors (72) on the adapter. In the embodiment shown, when fitted, the cover (60) abuts the distal surface (70) of the collar (62), although other means of securing the cover (60) to the collar (62) may be envisaged. In some embodiments, the cover may comprise a recess into which the collar is received, such that the collar sits inside the cover when the apparatus is assembled. The cover may be made from a soft, flexible material such as silicone, such that it is comfortable for the subject and pressure sore sensitive.

### Example 1: Testing the Phototherapeutic Effect

### Materials and Methods

### Organotypic Slice Culture Model

All animal procedures were licensed by the UK Home Office. An established in-vitro model for neurological damage sustained during TBI. The technique involved the sacrifice of an adult Sprague-Dawley rat (170-220 g) via hypercapnoea, from here the brain was extracted and both hippocampi extracted carefully. This was undertaken rapidly (within minutes) with regular bathing in glucose fortified neurobasal medium. Once extracted a Mcllwain (Camden instruments) tissue chopper was used to produce slices of 200 micrometers. The removal and slicing of the hippocampal tissue represents the injury stimulus to this model. The slices were then placed on semi permeable PTFE membrane inserts within 2 sets of standard 6 well culture plates, one set being for the purpose of ambient light control. From here 1 ml of clear media (glucose enriched neurobasal-A media, with added gentamycin) was then added and the plates were then placed in an incubator at 37° C. / 5% CO₂. An initial period of 4 hours within the incubator was observed to settle the organotypic slice culture. After this the culture trays were removed from the incubator and inspected for membrane adherence, and the first (day 1) dose of phototherapy applied. Initial efficacy trials commenced with an iridescence of 42 mW/cm² (as described below), and an exposure time of 2 minutes.

Phototherapy was delivered by a pre-calibrated enclosed LED array with a custom manufactured adaptor plate to house the 6 well incubation tray. The ambient light control was kept under identical conditions, however exposed only to ambient light during the 2 minute phototherapeutic intervention.

Cultures were maintained for 3 or 5 days. This relatively short period is focused on the clinically critical acute phase of injury (within the first 5 days after trauma).

It is hypothesised that the maximal benefits of direct to brain NIR phototherapy would be in the prevention of programmed cell death in metabolically distressed cells. NucView 488 is a caspase-3 fluorescent substrate, and a live cell imaging technique was used to identify cells entering programmed/apoptotic cell death. Caspase-3 is a key enzyme in the chain of events leading to apoptosis, and when activated it cleaves the NucView 488 substrate leading to the release of the fluorescent product. Therefore cells executing programmed cell death are clearly visible as discreet areas of fluorescence on microscopy, and serve as an effective method for quantifying the level of cell loss by apoptosis by counting the discreet areas of fluorescence on imaging key areas (dentate gurus, CA1 and 2) or the hippocampal slices (separate areas selected to account for differing levels of cell loss between different regions in the tissue). The apoptotic cell count from each region imaged serves as a single data point for comparison. Counting was undertaken by individuals in the research team blinded to the intervention.

### Retinal Ganglion Cell Survival

Retinal ganglion cell harvesting and culture is an established model of axonotemetic central nervous system, and serves as a useful parallel validation experiment to the organotypic slice culture model in ascertaining the potential benefits of direct to brain phototherapy. Here mixed adult rat retinal cultures containing enriched populations of Retinal Ganglion Cells (RGCs) were prepared from the same Sprague-Dawley rats as described above. In a similar technique to Ahmed et al. (Brain, 129(6), pages 1517-1533 (2006)), the retinal cells were dissociated using a Papain dissociation kit according to the manufacturer’s instructions (Worthington Biochemicals, Lakewood, NJ, USA). 125 x 10³ retinal cells/well were plated in poly-D-lysine and laminin pre-coated 8-well chamber slides and cultured in Neuronal Basal Medium (NBA). Cells were cultured for 5 days at 37° C. and 5% CO₂ as per the slice culture sections. One 8-well chamber slide was treated daily with 2 minutes of 660 nm light at the same iridescence and in parallel to the hippocampal slice model. A further 8-chamber slide was prepared and exposed only to ambient light in line with the slice culture control specimens. Specific adapters were constructed to mount the 8-well chamber slides on to the enclosed LED array to deliver the intended dosed of phototherapy.

Both the number of cells entering a programmed death within the organotypic slice culture model at 3 and 5 days, and the number of surviving retinal ganglion cells at 5 days were used to quantify the potential effectiveness of the direct NIR phototherapy. Non-parametric paired statistical assessments were used (Mann-Whitney-U) to identify statistically significant differences between the control and intervention samples|.

### Light Dose Characterisation

In order to ensure a consistent and adequate light dose delivery to the tissues a series of characterisation experiments were undertaken. The purpose of this was to ascertain the configuration of the source array in order to deliver an effective photonic dose precisely at the point in which the tissue slices or cells are in culture. A strong biphasic dose response exists, hence the importance of remaining within the therapeutic window must be born strongly in mind during experimental design.

A series of characterisation experiments were undertaken. The light source LED array was spectroradiometrically characterised using a National Institute of Standards and Technology (NIST) calibrated fibre coupled spectrophotometer (USB4000, Ocean Optics) to obtain information on absolute irradiance and wavelength.

A cosine corrector (4 mm diameter) attached to the optical fibre measured the irradiance delivered to the base of the membrane. The absolute spectral irradiance was recorded on OceanView software (Ocean Optics, UK) and the integral between 600-700 nm was taken to obtain absolute irradiance.

An irradiance of 42 mW/cm² was selected and the equipment configuration required to maintain this established (21.105 V and 1.24 amp supply to array).

### Results

### Quantification of the Phototherapeutic Effect

A total of 44 hippocampal slices (75 regional images) from 6 separate adult rat hippocampi (3 sacrificed animals) were set in culture for 3 days. Of these 24 were treated daily with 2 minutes of 42 mW/cm² 660 nm NIR light, and 20 were exposed to 2 minutes of ambient light only. The total number of discrete areas of cell loss to apoptosis were counted and compared between samples. The average number of apoptotic areas observed within the treated slices was 176 vs 234 (FIG. 4) in the untreated slices. This represents a significant reduction (24.7% reduction - Mann-Whitney single tailed; z score=-2.32, P= 0.02). Of the regions imaged 13 were incomplete due to artefact, and were discarded.

A total of 41 hippocampal slices (80 regional images from 6 separate adult Sprague-Dawley rat (170-220 g) hippocampi (3 sacrificed animals) were set in culture for 5 days. Of these 11 were treated daily with 2 minutes of 42 mW/cm² 660 nm NIR light, and 30 were exposed to 2 minutes of ambient light only. This discrepancy between groups was primarily due to the loss of a number of culture plates to fungal infection. The average number of discreet apoptotic areas seen in the slices receiving daily NIR therapy (5 days) was 1461 vs 1871 receiving only ambient light (FIG. 5). This represents a statistically significant reduction (21.9% reduction, Z score=-2.32009. The p-value is 0.01017). The large increase in observed apoptotic events at 5 days compared to those seen at 3 is likely to be due to the natural accumulation of cell death events in response to the initial stimulus.

In the parallel retinal ganglion cell culture experiment, a significantly higher rate of survival (50.2%) was observed in the 8 well chamber slide receiving daily 660 nm phototherapy (mean 824+/-52.9 per well vs 414+/-29.6 control, z= 3.203, p= 0.001). This represents an approximate doubling of the number of surviving cells from their original populations (FIG. 6). Since the severing of the optic nerve represents injury to the retinal cell axon strongly analogous to a spinal cord injury, these results demonstrate the potential benefits of direct low level light therapy to treat spinal cord injuries.

### Example 2: Device Construction

### Materials and Methods

### Adapter/Bolt Construction

A custom designed solid nylon threaded fibre adapter or ‘bolt’ was constructed (FIG. 7). Computer aided design was undertaken using Shapr3D (Techsoft 3D, Budapest Hungary).

### Light Source and Fibres

Four 30.5 mm diameter heat sink mounted light emitting diodes (LEDs) with a nominal wavelength of 660 nm and manufacturer stated power ratings of 940 mW (M660L4, Thorlabs UK) served as the light source. The LED chip size (emitter size) for each separate source was 1.5 mm x 1.5 mm. A stackable lens mount (SM1L10, Thorlabs UK) was attached to the LEDs and a SMA fibre adapter (SM1SMA, Thorlabs UK) and fitted directly above the LED chip to maximize coupling efficiency.

Each LED was individually powered using the manufacturer recommended LED driver (T-Cube LED driver, 1200 mA Max drive Current; LEDD1B, Thorlabs UK) and power supply (KPS101, 15 V. 2.4 A; Thorlabs UK). Four 1 m long SMA connected patch cables were used to transport light from the sources. The fibre diameters of these was 1500 µM and a numerical aperture of 0.5 (M107L01, Thorlabs UK). One end of each patch cable was cut under wet conditions across the cross section of the fibre. Subsequently, 5 cm of protective metal sheathing was removed and the cut end of the fibre was polished sequentially using 2000 grit and 4000 grit silicon carbide paper under wet conditions. The cut ends of the fibres were then rinsed and cleaned with 99% ethanol and inserted directly into the apertures of the intra-cranial access bolt.

### Light Source Characterisation

### Photodiode Power Meter

The absolute power of each LED source at the emitting surface and through the modified patch cables was determined using a relatively large sensor (10 mm) photodiode power meter (PD300 photodiode, Ophir Spiricon). The emitted radiant power of each LED (at the LED surface) and through the patch cable was measured with the light emitting area in contact or fixed ~ 0.5 mm from the external photodiode filter. Power output was recorded continuously for 30 s and peak power output was registered. The average power values (n=3) were used for optical correction of beam profile images.

### Spectral Irradiance

The spectral properties of each LED was measured using a UV-VIS spectrometer (USB4000, Ocean Optics, UK) at the LED surface and through the patch cable. The spectrometer was coupled to a 200 µm optical fibre and an opaline glass cosine corrector (CC3; 3.9 mm diameter), and radiometrically calibrated in compliance with National Institute of Standards and Technology (NIST) practices recommended in the NIST Handbook 150-2E against a traceable light source (Mikropack DH2000, Ocean Optics, USA). All spectra were collected in OceanView Software (Ocean Optics, UK) and analyzed to determine radiometric quantities.

### Results

A solid adapter in the form of a bolt was constructed from nylon using 3D printing (FIG. 7). Nylon was selected as it is readily available, inexpensive and has a high tensile strength. A rounded edge was created on the head of the adapter in order to prevent debris from falling from the thread edge and being deposited on insertion of the adapter. The bolt included six holes that pass all the way through the head and the screw thread. This included four 1.5 mm diameter holes for receiving the optical fibres, a fifth 2 mm hole for receiving a standard of care multi modal intra cranial monitoring probe, and a sixth 2 mm hole for receiving a further fibre for passing deep into the brain tissue.

FIG. 8 shows a graphical representation of the coupling efficiency between the LED source and the fibre tips installed within the face of the intra cranial access bolt intended to abut the surface of the brain. It can be seen that the combination of fibres and LEDs results in approximately 20-25% coupling efficiency. The total delivered power at the four fibres was 490 mW.

The decision to incorporate four primary delivery fibres was selected to test the initial the integrity of the bolt, in order to test mechanical stability during implantation. Even numbers of fibres are beneficial in order to allow paired spectroscopic examination of the brain tissue, for example by Raman or Near Infra-Red spectroscopy.

FIG. 9 shows the real time power (mW) measured over 30s exposures directly onto the PD300 photodiode power meter at the LED surface (FIG. 9A) and at the coupled fibre tip exit (FIG. 9B). It can be seen that over the 30s exposure, each channel produces stable outputs.

FIG. 10 shows the spectral irradiance of each channel measure at the LED surface (FIG. 10A) and at the coupled fibre tip exit (FIG. 10B). The peak wavelengths measured at the LED surface and at the coupled fibre tip exit was 667 nm.

For each individual source, the characterization data revealed the combination of LEDs and fibres result in a coupling efficiency of -20- 25%. This coupling efficiency may be due to one or more of the following factors:
The emitting area of the diodes was ~40% larger than the area of the fibre, such that not all of the emitted light was captured;
The power delivered into the fibres is dependent on the distance between the fibre and the LED due to divergence. Since electronic components and adapters restrict intimate contact between LED and fibre, the delivered power is reduced as a function of distance;
For surface emitting LEDs such as the M660L4 utilised here, the emission pattern is Lambertian which means the intensity is proportional to the cosine of the emission angle relative to the normal. The viewing angle of the LED is 120° but captured without cosine correction at 90° which can cause loss of power through divergence at angles greater the 90°;
Attenuation within fibres by absorption and reflection loss within the wall substance and at the LED coupled end.

The coupling efficiency may therefore be improved by one or more of the following:
The use of a laser, or increasing the fiber diameter, e.g. to match that of the LEDs;
The use of one or more lenses that converge the beam into a narrow point which is precisely located at the interface of the fibre;
Improving the diffuse reflectance within the LED-fibre attachment housing;
The use of bundled fibres.

### Example 3: Testing in a Phantom System

This experiment developed the concept of measuring irradiance within a given medium in order to effectively measure the dose radium (i.e. the radius of tissue or distance from the light source to which a therapeutic intensity of photons is delivered). The aim was to ascertain if light from the fibres mounted in the access bolt could be detected at a series of increasing distances through brain simulation liquid phantom media by a bare detection fibre connected to an optical power meter.

### Materials and Methods

A nylon adapter as described in Example 3 was used, and optical fibres were passed through the holes. The optode layout of the device was four multi-modal fibres (two used as source fibres and two used as detection fibres) organized in two opposing pairs, with a source detector separation of 11 mm and 90 degrees to each other. A standard of care catheter was placed equidistant between the two source fibres (these are however multimodal and interchangeable) and the geographical centre of the adapter. The adapter was ‘screwed’ into an acrylic skull, and firmly held the fibres in place with a watertight seal. After insertion there was no overt distortion or damage to the fibres and they remained functional. The standard of care/raumedic catheter was still easily removable and insert able after placement. An external depth limiting guide tube allowed the manufacturer’s recommended depth for the position of the raumedic catheter tip.

A ‘phantom’ liquid media was created using a lipid emulsion solution as previously described in the literature (Milej et al., Opto-Electronics Review, 18(2), pp208-213). This solution has established absorption and scattering properties similar to that of brain tissue. This solution was then poured into the acrylic skull and retesting of the phantom rig was undertaken to ensure the function of the source and detection fibres.

An Ocean Optics (Halma plc, India) broadband (white light) source was connected to the source fibres using a pair of SMA fibre optic connectors. An Ocean Opitcs ‘Flame’ spectrometer was then attached to the detector fibres. A proprietary MATLAB based software interface was used to observe the resultant detected spectra. For the purposes of the investigation each fibre pair (11 mm separation) was tested separately. Open testing (source on without any additional absorber or artifact) provided us with our baseline spectral reading for the phantom.

In order to observe the effect of an absorber at variable distances in front of the probe interface a 1 cm diameter black sphere was placed at the end of a rigid guide wire with accompanying slide measurement calipers. This allowed a precise measurement of the distance of the sphere from the probe face to be made.

### Results

No significant difference was seen in the detected broadband spectrum until the test sphere was within 2.4 cm of the probe face (FIG. 11A). After this a significant impact was seen on the recovered parameters (FIG. 11B). At approximately 3 mm distance very little of the emitted broadband spectrum was observed. Testing indicated that a single 30 mW source fibre produces an approximate irradiance of 1 mW/cm² at approximately 11 mm from the source fibre tip. The irradiance delivered may be increased by using a higher powered light source, by using more fibres and/or by using fibres of larger diameter.

This experiment demonstrates the device as a practical and functional temporary cranial implant, with a watertight secure platform that can house fibre optics and intra cranial monitoring catheters. The device remained functional after insertion and a consistent spectrum was observable from the detection fibres. The experiment further demonstrated the feasibility of detecting doses with the same optical fibres that are used as light sources. In addition, the results indicate that a distance of 0.3-2.4 mm between the probe face and the target tissue may be used to achieve physiological changes.

### Example 4: Cadaveric Testing

The device was tested on a fresh (non-embalmed) cadaveric specimen awaiting post-mortem at the University Hospital of Warsaw, Poland, Department of Clinical Pathology. The absence of formaldehyde preservative within the tissue is essential for realistic assessment of its optical characteristics.

After initial supine positioning of the cadaver, appropriate draping and covering was undertaken. As required for post-mortem investigation a bi-coronal scalp flap was opened. This exposed the majority of the frontal calvarium (skull) including the key area 11 cm posterior to the nation and 3 cm from the middling (Kocher’s point). This is the most common point in which invasive intra cranial monitoring catheters are inserted, due to the ineloquent nature if the underlying brain.

At Kocher’s point a single 14 mm burr hole was made with a clutched drill bit housed in a standard electrical drill. A durotomy (a cut in the dura) was made, this is also a standard part of the intra cranial access procedure. No further preparation of the hole was undertaken.

The bolt housing was then inserted by rotating in clockwise until the threaded operation of the bolt housing was no longer visible (8 mm). For the purposes of this provisional test the fibres were in situ during insertion.

The bolt (in situ) was then perturbed to the point where the weight of the head of the cadaver was supported by the bolt (this is frequently a bedside test used clinically to confirm the secure placement of a standard ICP bolt).

The LED array was then switched on to the same power levels as tested in the previous characterization experiments. A bare 1500 µM fibre similar to those employed in the bolt array itself was then used to measure light intensity through separate 5 mm holes in the skull at increasing distances from the edge of the inserted bolt.

Readings on four sides/aspects of the bolt at two distances were undertaken. In all cases the fibre was inserted into the brain parenchyma at 45 degrees towards the bolt source away from the brain surface perpendicular. Although this is not the optimal direction to represent the amount of light received by the cells in that specific region, (as it will significantly underestimate the total quantity), due to limitations on the quantity and size of holes permitted this was the best observation angle possible.

### Results

Results indicated that at a distance of 2.5 cm on all sides measured (indicating irradiation of a nominal volume of 50 cm², assuming equal measurements would be obtained from 2.5 cm directly below the probe face) a power of 0.5 mw/cm² was observed (FIG. 12). This measurement will represent a fraction of the actual true dose due to the placement angle of the measurement fibre as explained above.

In our investigations into efficacy, the approximate optimal dose was observed as 42 mW/cm². However, at half of this dose we recorded very nearly the same reduction in cell loss. Therefore a broad window of effective phonic dose exists, which is important to consider when planning the eventual power and light source positional distribution.

The cadaveric evaluation we have undertaken indicates that a dose of approximately 10% of the irradiance/intensity required for a significant reduction in cell loss in 21 cm³ of brain tissue (or approximately 8% of the volume of the entire hemisphere) was achieved with the equipment tested.

In order to achieve the desired therapeutic dose, the light source or beam properties may be modified to improve coupling efficiency, light delivery and homogeneity, in one or more of the following ways:
Using high-powered light sources such as higher-powered LEDs or lasers at appropriate wavelengths (e.g. 660 nm)
Increasing the fibre diameter (e.g. to 2000 µm)
Using bundled fibres
Spacing the fibres evenly and providing overlapping beam profiles
Use of optics such as lenses and/or diffusers
Selection of adapter material to improve scatter and reflection
Altering the angle of the fibres.

### Example 5: Monitoring Treatment Using Raman Spectroscopy

As a means of observing tissue health during and after light therapy treatment, the fibre optic interface used to deliver the light therapy may be used to optically monitor the tissue using Raman and/or near-infrared spectroscopic examination during periods where therapeutic light is not being delivered (the vast majority of the time).

Critically - changes in these spectra could be used to confirm the effects of the NIR therapeutic light, giving a clear indication that the tissue has ‘benefitted’ from the therapy. In order to examine the possibility of this being utilised as a viable bio-feedback mechanism for direct-to-brain NIR therapy we performed a further series of experiments in organotypic tissue.

### Materials and Methods

In the manner as previously described in Example 1, a 3-day organotypic hippocampal slice culture was performed (40 separate slices). Daily treatments (one treatment per day) of using an irradiance of 42 mwW/cm² irradiance at 660 nm light for 1 minute were applied to 20 of these, with the remaining 20 slices receiving ambient light as an equivalent control.

After the 660 nm treatment the cultured hippocampal slices were removed from their semi-permeable membranes and placed onto a standard mounting slide covered in standard aluminium foil, then placed into a Renishaw inVia Qontor (UK) Raman spectrometer, with a 633 nm laser source. Once focused onto the surface of each tissue slice a Raman spectrum was obtained and normalised to cosmic background radiation.

The spectra acquired from light therapy treated slices was then compared to those that had received only ambient light.

### Results and Discussion

FIG. 13 shows the Raman spectrum of the control versus the irradiated tissue. A significant shift in the ratio/morphology of the 1440 and 1660 cm⁻¹ was observed (FIG. 14).

Our investigation into the effect of 660 nm low level light therapy on the Raman spectroscopic signature on rat brain tissue demonstrates a clear and consistent change in ratio bands at 1440/1660 cm⁻¹ (FIG. 14). This observation indicates that not only will it be possible to monitor the quantity of light delivered but the initiation of the cell saving effect itself could potentially be feasible.

Importantly, even within this small data set of 40 organotypic slices (20 control and 20 intervention) a significant difference in 1440-1660 nm spectrum can be seen (FIG. 13). These areas are specifically related to key enzymes in the oxidative metabolic electron transport chain.

This demonstrates that the array could allow a comprehensive biofeedback mechanism which can meter the precisely required photon dose to a specified volume of brain tissue, and which can detect specifically when the tissue has responded to the dose.

### Example 6: Dose Modulation Using Feedback Loop

In order to deliver an optimal dose to any given target tissue at a specific distance from the optical interface, a table of target tissue intensity can be developed using a suitable model of the target tissue, for example a brain tissue phantom model. This model takes the form of a lipid suspension fluid set with gelatine, with a chromophore added to provide optical character which is equivalent of brain-equivalent i.e. having equivalent coefficients of absorption and scatter for brain tissue which are known values.

Effective real-time feedback can then be developed by formulating sets of known tissue intensity/irradiance tables. With reference to FIG. 15, measuring light intensity within the phantom tissue at increasing distances from the optical source / detection interface- with one or more source fibres (A) emitting light, and one or more detection fibres (B) receiving light/photons returning through the tissue (banana shaped path) will allow the generation of these tables.

Through this method a given target intensity, to a given region of tissue at a given distance from the source/detector array may be achieved by modulating the light sources depending on the light intensities received via the array detection fibres.

### Example 7: Beam Profile Characterization

### Materials and Methods

### Standardized Moulds

Custom PLA moulds were designed and 3D printed to allow for a standardized methodology for the assessment of beam profile. The moulds had an inner diameter of 50 mm, an outer diameter of 60 mm and centrally M14 threaded through hole of 14 mm with a depth of 15 mm to allow for light delivery using standard dimension ‘photobolts’. Each mould was fabricated with increasing heights of the outer wall which allowed testing of phantom media between 0, 2.5, 5, 10, 15 and 20 mm.

### 3D Printed Photobolts

A series of 3D printed PLA photobolts (i.e. adapters) were made which consisted of 7, 14, 21 or 28, 1000 µM uniformly spaced through holes centrally in the bolts. The holes were subsequently drilled using a 1000 µM hand-drill to remove the internal debris from 3D printing.

### Phantom Model

Phantom media was prepared using 95% full fat milk, 5% Porcine Gelatine (Sigma Aldrich) and 200 µL blue ink. A photobolt was then inserted into the apertures of the standardised moulds and the phantom media was poured directly in. The phantom media was left to set for 1 hour before testing.

### Light Source and Fibers

Four 30.5 mm diameter of heat sink mounted LEDs with a nominal wavelength of 660 nm and manufacturer stated power ratings of 940 mW (M660L4, Thorlabs UK) served as a source. The LED chip size (emitter size) for each separate source was 1.5 mm x 1.5 mm. A stackable lens mount (SM1L10, Thorlabs UK) was attached to the LEDs and a SMA fiber adapter (SM1SMA, Thorlabs UK) and fitted directly above the LED chip to maximize coupling efficiency. Each LED was individually powered using the manufacturer recommended LED driver (T-Cube LED driver, 1200 mA Max drive Current; LEDD1B, Thorlabs UK) and power supply (KPS101, 15 V. 2.4 A; Thorlabs UK). Four 1 m long custom SMA connected patch cables having 7 x 1000 µM diameter strands (Thorlabs, UK) were used to transport light from the sources to the lower surfaces of the phantom media. The data was compared with single solid core (1500 µM; numerical aperture of 0.5; M107L01, Thorlabs UK). For the single core solid fibers, SMA connection on one end of each patch cable was removed by cutting under wet condition across the cross section of the fiber. Subsequently, 5 cm of protective metal sheathing was removed and the cut end of the fiber was polished sequentially using 2000 grit and 4000 grit silicon carbide paper under wet conditions. The cut ends of the fibers were then rinsed and cleaned with 99% ethanol and inserted directly into the apertures of the intra-cranial access bolt.

### Light Source Characterisation

### Photodiode Power Meter

The absolute power of each LED source at the emitting surface and through custom patch cables was determined using a relatively large sensor (10 mm) photodiode power meter (PD300 photodiode, Ophir Spiricon). The emitted radiant power of each LED (at the LED surface) and through the patch cable was measured with the light emitting area in contact or fixed ~0.5 mm from the external photodiode filter. Power output was recorded continuously for 30 s and peak power output was registered. The average power values were used for optical correction of beam profile images.

### Spectral Irradiance

The spectral properties of each LED was measured using a UV-VIS spectrometer (USB4000, Ocean Optics, UK) at the LED surface and through the patch cable. The spectrometer was coupled to a 200 µm optical fibre and an opaline glass cosine corrector (CC3; 3.9 mm diameter), and radiometrically calibrated in compliance with National Institute of Standards and Technology (NIST) practices recommended in the NIST Handbook 150-2E against a traceable light source (Mikropack DH2000, Ocean Optics, USA). All spectra were collected in OceanView Software (Ocean Optics, UK) and analyzed to determine radiometric quantities.

### Beam Profile

A silicon based charge coupled device (CCD) camera beam profiler (SP620, Ophir, Spiricon, Israel) was used to measure the spatial distribution of irradiance for each standardized mould and adaptor combination. The camera and 50 mm CCTV lens (Ophir, Spiricon, Israel) were separated by 30 mm spacer rings between the CCD sensor and lens, and focused onto targets at 200 mm distance. An optical scaling calibration was applied by measuring a sample of known dimensions and calculating a scaling factor which enabled pixel dimension correction in the plane of the targets. This process enabled precise linear measurement of the images and accurate beam diameter determination. The camera was further equipped with a combination of neutral density filter (Ophir, Spiricon, Israel) to avoid CCD sensor saturation. and an international corrective filter (International Light Technologies, US) to correct for spectral response of the CCD sensor.

Prior to beam imaging in each measurement, sensor saturation levels were controlled by a combination of neutral density filters, the internal aperture of the CCTV lens or by adjustment of the camera integration time. Once saturation levels were optimized, the system was corrected for ambient light and pixel response using the UltraCal function in Beam Gage Software (Ophir, Spiricon, Israel). Subsequently, each image was individually calibrated, according to the power recorded from the photodiode power meter. Thus the images were calibrated by setting the highest level measured to assume the highest array count in BeamGage Professional (v5.6: Ophir Spiricon) software. The diameter of the active light beam (D4σ, ISO Reference 11145 3.5.2) was determined automatically by the Beam Gage software and was used to determine the irradiance scale on each image based on the inputted power values.

### Results and Discussion

FIG. 16 shows the beam profile distributions measured through phantom media in standardised moulds of thickness between 0-20 mm. With an increasing number of bundled fibers compared with a solid core fiber, the beam distribution is shown to be more uniformly distributed having a larger optical footprint. These effects are more pronounced at lower thicknesses of phantom media. However, both power and irradiance exponentially decrease with increasing thickness of phantom media (FIG. 17) irrespective of the number of fibers. Whilst this is the case, an optical window of effective irradiances has been identified with the current combination of light sources and fibers. This is represented by the shaded area in FIG. 17B, which demonstrates that irradiances which are within the threshold values of effective doses (16-80 mW/cm²) can be delivered within 10 mm depths of brain tissue and perhaps at greater depths with 4x multicore fibers. In addition, since the multi-cored fibers have significantly increased beam distribution and beam width and beam flatness, these effects are likely to be more pronounced volumetrically rather than in any one direction individually (i.e. either through depth or spatially across the surface individually). Further improvements are possible by using higher powered lights sources or lasers, sources with larger optical footprints and utilising a greater number of sources and fibers. In addition, for even deeper beam penetration, a longer wavelength such as 810 nm or greater could be used since scattering and absorption by brain tissue components may be reduced.

### Example 8: Evaluation of Low Level Light Therapy (LLLT)

The aims of this work were to:
1. Evaluate the potential of LLLT in reducing the quantity of brain tissue cells lost to apoptosis in a hippocampal organotypic slice culture model (as an in vitro TBI model);
2. Investigate the influence of irradiance (photon density), the effect of irradiance and exposure time (total photon dose), and consecutive daily doses on the number of cells initiating programmed cell death within the cultured tissue; and
3. Investigate changes in tissue optical (Raman) spectroscopic signature elicited by LLLT along with its relation to photon dose.

### Materials and Methods

### Sprague-Dawley Rat Hippocampus Organotypic Slice Model

A hippocampectomy was performed immediately post-mortem on adult Sprague-Dawley rats under Home Office licence, sacrificed via carbon dioxide toxicity. Once performed, 150 µm hippocampal slices were separated. Individual slices were then placed onto polytetrafluoroethylene (PTFE) semi-permeable membranes (4 slices per membrane) incorporated into individual well inserts (Millicell cell culture inserts, Millipore, PICM03050) fitting into a standard 6 well culture plate (Sigma, SIAL0516). Glucose fortified B27-supplemented neurobasal medium (sNBA) solution (1 ml) was then added to each well. Plates containing the slices were light protected using foil and incubated at 37° C. / 5% CO₂. The initial dissection and slicing of the tissue, particularly the manual slice separation serves as an in vitro analogue of TBI, not requiring the stable culture population and injury calibration of previously described ‘stretch’ models. A daily visual assessment was undertaken. All subsequent analysis would be carried out on tissue and media harvested from this process, paired control and intervention samples were obtained from the same sacrificed specimens.

### Photon Dose Delivery, Beam Profile and Calibration

Therapeutic light of 660 nm wavelength was delivered in a single daily dose using a variable irradiance (power) calibrated LED light source array (BioThor device, Thor Photobiomedicine). Light intensity was controlled using a variable voltage power supply and irradiance and beam homogeneity were confirmed via UV Vis Spectroscopy and beam profiling respectively.

### Spectrophotometric Light Characterisation

The BioTHOR plate irradiator was spectroradiometrically characterised using a National Institute of Standards and Technology (NIST) calibrated fiber coupled spectrophotometer (USB400 UV-Vis Spectrometer, Ocean Optics) to obtain information on absolute irradiance and wavelength (FIG. 18). Prior to calibration, the spectrometer was assembled with a 200 µM optical fiber and CC3 opal glass cosine corrector (3.9 mm diameter). Following calibration, an empty 6-well plate was placed into the plate carrier with an aluminium ‘mask’ directly below the plate so only the area corresponding to the wells were exposed to light. The cosine corrector of the calibrated fiber was placed centrally into each well of the 6-well plate so that the surface of the cosine corrector was in contact with the lower surface of the well to allow the measurement of the amount of light delivered during in vitro irradiation of hippocampal slices. Adjustment of the supply voltage to the array to pre-determined values supplied by the manufacturer allowed specific irradiances to be delivered to the plates. The spectral irradiance was recorded using Ocean View software (Ocean Optics, UK) and the absolute irradiance was calculated from the integral of the emission trace.

### Beam Profile Measurements

The beam profile of the BioTHOR plate irradiator was determined using a CCD based beam profiler camera (Spiricon SP620, Ophir) following optical and linear calibration. The camera was focused onto the clear lower surface of a 6-well plate and beam profiles were measured either with or without a diffuser target screen (Opal glass target screen, Thorlabs) placed between the 6-well plate and light source. For each measurement an ambient light correction was applied and images were recorded statically to assess the homogeneity of the beam delivered in each well.

### Dosing

A commencing dose of 2 minutes irradiance at 42.4 mW/cm² daily was selected. The six well plate containing slices to be treated (intervention plates) was placed on the source, covered over with an ambient shield. Control plates containing hippocampal slices from the same sacrificed animals (identically and contemporaneously prepared and incubated adjacent to the intervention plates) were placed in ambient light during treatment. All plates were foil shielded as soon as the light therapy was completed and then returned to the incubator.

### ImmunoFluorescent Cell Imaging

NucView 488 Caspase-3 was utilised as a fluorescent apoptotic cell marker. Caspase-3 is a key enzyme within the apoptotic pathway, and once activated (as apoptosis is triggered) it cleaves the NucView 488 substrate liberating the fluorescent product. On the final day of culture, the media within each well was replaced with 1 ml of media containing Nucview 488 (1/200 dilution e.g. 5 µl NucView in 995 µl glucose fortified sNBA). Plates were then incubated for a further 4 hours. The well media was then replaced with 1 ml 4% paraformaldehyde (PFA) and incubated for 20 minutes shielded from light at room temperature. The membrane inserts containing hippocampal slices then underwent three consecutive washes in phosphate buffered saline (5 minutes per wash). These were then removed from their respective wells and mounted using VECTASHIELD antifade mounting medium containing 4,6-diamidino-2-phenylindole (DAPI) for the purpose of additional discreet nuclear marking. A Zeiss axioscope was used for the purpose of fluorescent imaging. Three specific and consistently recognisable regions were imaged on each hippocampal slice, the dentate gyri, and the coru ammonis (CA) regions 1 + 2. Three images were obtained for each region without Z stack: an image showing the total number of cells (DAPI fluorescent blue channel); an image showing only the apoptotic cells (NucView 488 related green channel); and a merged image to observe the relative ratio of these. Images were discarded where image quality was deemed poor or corrupted with artefact or inclusion.

For live cell imaging experiments (investigating the effect of consecutive daily LLLT doses), imaging took place daily. Media was replaced each day with 1 ml media containing the Nucview 488 and pure DAPI (5 µl NucView and 1 µl pure DAPI in 994 µl sNBA). Plates were then incubated at 37° C., 5% CO₂ for 4 hours. Following incubation, images were taken as per the above protocol, however without removal of the membranes or fixation. The fluorescent media was then removed and replaced with sNBA. As a contrast agent for cell nuclei was not applied, only absolute numbers of cells entering (caspase-mediated) apoptosis are obtained. The next LLLT dose was then applied where applicable. Plates were then returned to the incubator (37° C., 5% CO₂).

Images obtained were then analysed and automatic cell counts acquired using ImageJ (NIH, University of Wisconsin, USA). For each slice in all three regions (DG, CA1, CA2), the total cell numbers (DAPI blue channel fluorescence) and apoptotic cells (NucView) were counted providing a percentage ratio of cells that had initiated apoptotic cell death.

### Tissue Raman Spectroscopy

Immediately after the final LLLT or control treatment in each individual culture experiment individual slices retrieved from the culture wells were placed onto an aluminium backing plate (optical noise reduction) and gentle pressure exerted on the tissue sample directly to create a (visually) homogenous and uniformity thick Raman scanning surface. Spectroscopy was carried out using an inVia™ confocal Raman microscope with incorporated spectrometer (Renishaw, Wotton-under-Edge, UK), The integrated ‘WiRE’ software package (Renishaw, Wotton-under-Edge, UK) was used for acquisition and image processing. After surface focusing using 20x objective magnification a 633 nm laser at 100% device specific power was used to obtain spectra. A total of 3 x 6 second exposures were obtained to formulate the definitive spectrum from each hippocampal slice. Acquisition and processing were carried out in line with previous investigations undertaken (Banbury, C. et al. Sci. Rep. 9, 1-9 (2019), Banbury, C. et al. Biomed. Opt. Express 11, 6249-6261 (2020)). The peak intensity at 1440 cm⁻¹ and 1660 cm⁻¹ were the focus of analysis along with their respective ratios (due to expected variability in raw data absolute quantity).

### Statistical Analysis

Data was assessed for normal distribution (Shapiro-Wilk) and resultantly where data was non-parametric, a Mann-Whitney statistical test was used to ascertain the significance between the two considered continuous variables (intervention and control), and the student t-test in the case of normally distributed data. A P value < 0.05 was considered statistically significant within this context. Other statistical tests (Tukey HSD) were also employed for multiple comparisons where applicable.

### Results

### 1) Initial Effect

An initial control group of 28 slices (n = 82 observed regions of interest (ROI)) was compared with a light irradiated group of 40 slices (n = 95 ROI) from a total of 5 sacrificed animals. An initial 2 minutes of LLLT at was applied daily for 5 consecutive days at an irradiance of 42.4 mW/cm² (LT2). On average, significantly more apoptotic cells 62.8± 12.2% vs 48.6± 13.7% (P<0.0001) per region were observed in the control group compared with the LLLT irradiated group (FIG. 19). The individual slice with the highest percentage of apoptotic cells (85.6%) occurred within the control group. Conversely, the irradiated group contained the slice with the lowest percentage of apoptotic cells (14.1%). A differential benefit (in terms of proportion of cells lost to apoptosis) was also observed between individual hippocampal regions (FIG. 20).

### 2) Effect of Irradiance

Subsequently, 5-day cultures with daily (2 minute) treatments at an irradiance of 21.3 mW/cm² (LT1) and 85.0 mW/cm² (LT3) were then undertaken in a total of 20 and 24 hippocampal slices (n = 57 and 67 regions, respectively) along with 18 contemporaneous control slices (n = 35 regions) from 4 sacrificed specimens. Within the LT1 treated slices 45.8±12.7% of observable cells underwent apoptosis, and significantly less than the 62.8±12.2% observed in the control specimens (FIG. 21; P < 0.0001). Within the LT2 treated slices the observable apoptotic cell population was 43.9± 14.6% and 54.7±11.9% in the LT3 group, both significantly lower than control (FIG. 3; P = 0.0018 and P < 0.0001, respectively).

### 3) Effect of Dose Duration

Daily of doses of 1 minute, 2 minutes (initial) and 3 minutes were then applied to the 5-day slice culture model. In total 14 slices (n = 41 ROI) received 1 minute of irradiation a day, 14 slices received 2 minutes (n = 39 ROI) and 15 slices (n = 44 ROI) 3 minutes. The previously ascertained optimal intensity of 42.44 mW/cm² was applied, with a contemporaneous control group of 15 slices (n = 43 ROI) cultured from a total of 3 sacrificed specimens. The mean proportions of apoptotic cells observed in control slices was 60.2±11.0% compared with 51.6±17.0% in the 1 minute group (P= 0.0066), 54.6±13.5% in the 2 minute group (P= 0.0437), and 61.4±15.2% in the 3 minute group (P = 0.182) (FIG. 22).

### 4) Cumulative Effect of Daily Doses

A simultaneous 5-day culture with daily live imaging was undertaken with: (1) a control group of 10 slices (no light therapy, n = 27 ROI), a 1 day group of 10 slices (receiving one dose of light therapy 0 h after injury and no further doses, n = 27 ROI), a 2 day group of 10 slices (receiving two doses of light therapy at 0 h and 24 h after injury then no further doses, n = 30 ROI), a 3 day group of 9 slices (receiving three doses of light therapy at 0 h, 24 h and 48 h after injury and no further doses, n = 25) and a 4 day group of 10 slices (received four doses of light therapy at 0 h, 24 h, 48 h and 72 h after injury, n = 28 ROI).

The control group of slices showed a steady reduction in apoptotic cell death over the 4-day experiment (Table 1). After 2 days, no group of cultured slices demonstrated a significant reduction in apoptotic cells compared with the control. At 3 and 4 days, intervention groups had significant apoptosis reduction compared with the control group (P = 0.039 and P = 0.008 respectively). A cumulative beneficial effect was demonstrable (FIG. 23) on daily doses.

TABLE 1

Proportion of apoptotic cell loss (%) at 24, 48, 72 and 96 hours in control (no LLLT) and 1, 2, 3 and 4 consecutive days of LLLT.

| | 24 hrs | 48 hrs | 72 hrs | 96 hrs |
|---|---|---|---|---|
| Control | 67.4±6.10% | 64.7±4.02% | 63.6±5.60% | 62.4±4.00% |
| 1 day | 62.6±15.2% | 62.1±5.05% | 61.3±3.39% | 60.4±3.84% |
| 2 day | 64.2±5.20% | 59.2±8.59% | 60.2±3.09% | 59.7±3.84% |
| 3 day | 62.2±6.84% | 58.6±5.54% | 56.7±6.05% | 56.8±3.83% |
| 4 day | 64.7±7.73% | 61.8±4.55% | 57.1±5.80% | 54.7±5.27% |

### 5) Raman Spectroscopic Signature of Effect

After a 5-day organotypic culture, 36 slices underwent Raman spectroscopy (18 control and 18 receiving LT1 irradiance therapy for 1 minute a day). For each of the spectra produced, the shift corresponding to the largest peaks in the control condition were 1440±0.7 cm⁻¹ and 1659±0.8 cm⁻¹ In the treatment condition, they were at 1440±0.6 cm⁻¹ and 1658±0.6 cm⁻¹. For brevity, the peaks in both groups were approximated to 1440 cm⁻¹ and 1660 cm⁻¹, respectively. Focus centred chiefly on the magnitude of shift at these points in the acquired Raman spectra (FIG. 24).

Average intensity (peak size) at 1440 cm⁻¹ was 1407±187 (au) and 1288±110 in the control and LLLT conditions, respectively. The 1660 cm⁻¹ peak had a mean intensity of 1090±130 in the control samples, and 1291 ±66 in the treatment samples (FIG. 24). There was a significant change in the average 1440/1660 cm⁻¹ peak ratio between the therapy and control groups (Mann-Whitney U, P = 0.0204), with the ratio increasing from 0.774 in the control group to 1.002 in the therapy group (24.8% relative increase in ratio).

An additional cohort of 14 slices was prepared and 7 of these underwent a 3-day slice culture with daily LLLT at LT1 irradiance for 1 minute prior to Raman analysis, with the remaining 7 slices progressing to a 5-day (daily 1 minute LT1) culture before spectroscopic examination in order to observe the progression of the ratio over multiple daily doses. This culture was undertaken with an identical number of matched control slices. Here, an increase in 1440 cm/1660 cm⁻¹ ratio from the 3 day culture samples vs controls (0.757 vs 0.891; 17.7% increase, P = 0.092) was observed, along with a further increase in the ratio after 5 days. No significant difference was observed between the 1440 cm/1660 cm⁻¹ peak ratios at each therapeutic interval (3-day vs 5-day; P = 0.4).

### Discussion

Irradiance with 660 nm light has demonstrated a significant improvement in cell survival. An exposure of 1 minute at an irradiance of 42 mW/cm² had the greatest decrease in the number of apoptotic cells (an absolute reduction of 18.9%). When assessing the effect of the duration of exposure, 1 minute demonstrated the greatest magnitude of reduction in programmed cell death. From the observations on intensity and duration, we can infer that a certain rate of delivery and/or a total number of absorbed photons will exert the greatest positive effect.

Live image daily assessment demonstrated a positive cumulative effect of daily application of 1 minute LLLT at the optimally established irradiance of approximately 42 mW/cm². Daily application led to a progressive reduction in programmed cell death. The reduction in apoptosis within the live daily imaged control samples (reduction in live cell stock within the organotypic slice culture translating to a lower absolute number) was further decreased by the application of daily doses of LLLT.

The Raman spectra recovered and compared between both control and intervention slices revealed a clear signature of tissue response to LLLT (as tested at 42 mW/cm² intensity for 1 minute per day). All characteristic peaks identified and assigned in Table 2 are larger in magnitude for the average spectra of LLLT samples than control, representing a greater number of protein, lipid, amino acid and glycogen molecular bonds, indicating fewer apoptotic cells. Due to Raman intensity having arbitrary units, peak heights between separate samples are not an ideal source of comparison, thus the most comparable feature in these spectra is the ratio between the largest peaks at 1660 cm⁻¹ and 1440 cm⁻¹. In LLLT samples, the ratio between these peaks is closer to 1, whilst the 1660 cm⁻¹ peak decreases at a greater rate than the 1440 cm⁻¹ peak in control samples. The small decrease in intensity at 1440 cm⁻¹ indicates the potential reduction of metabolic processes with fewer cholesterol bonds. A greater decrease in the 1660 cm⁻¹ peak, in the Amide I region, suggests a reduction in the number of protein bonds in the tissue and increased apoptosis. Comparison between LLLT tissue and healthy tissue may therefore be used to quantify the extent of apoptosis prevention of LLLT following TBI.

Spectroscopic changes thus provide a potential avenue for real time monitoring of the effects of LLLT within the in vivo and/or clinical setting. Observing a shift in peak ratio may provide insight into when an optimal photon dose of LLLT has been delivered, removing the risk of over or under dosing leading to a reduction in potential positive effect. Secondly, this shift in 1440/1660 cm⁻¹ ratio may provide an independent measurement of brain injury burden and prognostic potential, allowing a real time roadmap of tissue recovery together with information regarding the effectiveness of other therapeutic interventions.

TABLE 2

Raman peak assignment for characteristic peaks identified in Rat Hippocampi Spectra

| Peak Wavenumber (cm⁻¹) | Assignment | Origin |
|---|---|---|
| 1004 | v(C-C) ring | Phenylalanine |
| 1063 | v(C-C) lipids | Phospholipids, Aliphatic side chains |
| 1129 | v(COC), Vₛ(PO_{I}), v(CN), V(CC) | Glycogen, DNA, Phenylanine, Lipids, Aliphatic side chains |
| 1209 | vPh, 5CHC, CH₃ sagging and twisting | Hydroxyproline, Tyrosine, Tryptophan, Phenylalanine |
| 1263 | Arnide III, CH₃ twisting | Lipids |
| 1297 | δ(CH₂) lipids, Amide III | Aliphatic side chains, Lipids |
| 1440 | δ(CH₂) lipids and proteins, CH₂ twisting and bending | CHolesterol, Phospholipids, Tyrosine, Proteins |
| 1660 | v(C=C) lipids, C=C stretching Amide I | Tyrosine, Lipids, Proteins, Alpha-helix/random coil |

v= stretching vₛ = symmetric stretching δ = in-plane bending, Ph = Phenyl

1-48. (canceled)

49. An apparatus for treating TBI or TSI in a subject, the apparatus comprising:
at least two optical fibers;
a light source which is operatively connected to at least one of the optical fibres; and
an adapter for securing the optical fibres to the body of the subject.

50. The apparatus according to claim 49, wherein the adapter is configured to secure the optical fibres to the body such that light from the light source is delivered directly to the brain or spinal cord, when the apparatus is in use.

51. The apparatus according to claim 49, wherein the adapter comprises at least one hole or slot for receiving the optical fibres therethrough.

52. The apparatus according to claim 49, wherein the adapter comprises a securing element for releasably attaching the adapter to the body, wherein the securing element comprises a screw thread or an adhesive portion or a loop or aperture.

53. The apparatus according to claim 52, wherein the adapter comprises a collar for receiving the optical fibres therethrough, the collar being attached to, mounted on or integrally formed with the securing element.

54. The apparatus according to claim 49, wherein the adapter further comprises a locking element for locking the position of the optical fibres relative to the adapter.

55. An apparatus for treating TBI or TSI, the apparatus comprising:
at least two optical fibres; and
a light source which is operatively connected to at least one of the optical fibres,
wherein the apparatus is configured to deliver light at an irradiance of from 10 to 90 mW/cm² directly to the brain or spinal cord.

56. The apparatus according to claim 49, wherein at least one of the optical fibres is a delivery fibre for delivering light, the delivery fibre being connected to the light source, and at least one of the optical fibres is a detection fibre for detecting the amount of light received by the brain or spinal cord in use, wherein the detection fibre not connected to the light source.

57. The apparatus according to claim 56, further comprising one or more spectrometers or a power meter to which the detection fibre(s) is(are) connected.

58. The apparatus according to claim 49, further comprising a controller.

59. The apparatus according to claim 58, wherein at least one of the optical fibres is a delivery fibre for delivering light, the delivery fibre being connected to the light source, and at least one of the optical fibres is a detection fibre for detecting the amount of light received by the brain or spinal cord in use, and the detection fibre not connected to the light source wherein the controller is configured, in use, to modulate the light delivered to the brain or spinal cord by the delivery fibres in response to the light detected by the detection fibres, such that a desired dose is delivered.

60. The apparatus according to claim 58, wherein at least one of the optical fibres is a delivery fibre for delivering light, the delivery fibre being connected to the light source, and at least one of the optical fibres is a detection fibre for detecting the amount of light received by the brain or spinal cord in use, and further comprising one or more spectrometers or a power meter to which the detection fibre(s) is(are) connected, wherein the controller is configured, in use, to modulate the light delivered to the brain or spinal cord by the delivery fibres in response to a spectrum obtained by the spectrometer.

61. A method for the treatment of TBI or TSI, the method comprising the direct delivery of light to the brain or spinal cord.

62. The method according to claim 61, wherein the method comprises delivering an irradiance of from 10 to 90 mW/cm², optionally an irradiance of from 20 to 60 mW/cm² (e.g. about 42 mW/cm²).

63. The method according to claim 61, wherein the light is delivered using an apparatus according to claim 1.

64. The method according to claim 61, wherein the method further comprises monitoring the light received by the brain or spinal cord tissue, and determining whether a sufficient or optimal dose has been delivered to the tissue.

65. The method of claim 64, wherein the method further comprises modulating the light delivered so as to achieve a desired dose, optionally in real time.

66. The method according to claim 61, wherein the method further comprises determining the effectiveness of treatment.

67. The method according to claim 66, wherein determining the effectiveness of the treatment comprises detecting a change in a spectrum obtained from the treated tissue with a reference spectrum.

68. The method according to claim 66, wherein determining the effectiveness of the treatment comprises determining the 1440/1660 cm⁻¹ ratio in a Raman spectrum obtained from the treated tissue.
